(19) United States
(12) Patent Application Publication
(10) Pub. No.: US 2017/0232140 A1
(43) Pub. Date: Aug. 17, 2017

(54) ADHESIVE COMPOSITIONS AND PATCHES, AND ASSOCIATED SYSTEMS, KITS, AND METHODS

(71) Applicant: Xcede Technologies, Inc., Rochester, MN (US)

(72) Inventors: Daniel Grant Ericson, Rochester, MN (US); Kyle Robert Brandy, New Hope, MN (US)

(73) Assignee: XcedeTechnologies, Inc., Rochester, MN (US)

(21) Appl. No.: 15/502,545

(22) PCT Filed: Aug. 7, 2015
(86) PCT No.: PCT/US2015/044225

§ 371 Date: Feb. 8, 2017

Related U.S. Application Data
(60) Provisional application No. 62/035,250, filed on Aug. 8, 2014.

(51) Int. Cl. A61L 24/10; A61L 24/08; C09J 133/02; C09J 7/02; A61L 24/00; A61L 24/06

(52) U.S. Cl. CPC A61L 24/106; A61L 24/0042; A61L 24/06; A61L 24/0094; C09J 133/02; C09J 7/0239; A61L 24/0005; A61L 24/08; A61L 2300/418; A61L 2400/04; A61L 2400/06; C09J 2489/006; C09J 2433/00; C09J 2403/006

(57) ABSTRACT

Adhesive compositions and patches, and associated systems, kits, and methods, are generally described. Certain of the adhesive compositions and patches can be used to treat tissues (e.g., in hemostatic or other tissue treatment applications), according to certain embodiments.

### RELATED APPLICATIONS

This application claims priority under 35 U.S.C. §119(e) to U.S. Provisional Patent Application Ser. No. 62/035,250, filed Aug. 8, 2014, and entitled “Adhesive Compositions and Patches, and Associated Systems, Kits, and Methods,” which is incorporated herein by reference in its entirety for all purposes.

### TECHNICAL FIELD

Adhesive compositions and patches, and associated systems, kits, and methods, are generally described.

### BACKGROUND

Hemostatic agents and tissue sealants are routinely used to prevent excess blood loss and to reconstruct tissue during surgical repair. Fibrin glue was approved by the FDA in the 1990's and can be used to impart topical hemostasis, provide sealant properties that are suitable is some clinical applications, and promote tissue approximation. Fibrin glue mimics the final steps of the coagulation cascade. In the presence of thrombin, fibrinogen is converted to fibrin. Thrombin also activates Factor XIII, which stabilizes the clot, by promoting polymerization and/or cross-linking of the fibrin chains to form long fibrin strands. This process usually occurs in the presence of calcium ions. It proceeds independently from the remainder of the coagulation cascade, and provides some degree of hemostasis even with defects in other portions of this pathway. There is subsequent proliferation of fibroblasts and formation of granulation tissue within hours of clot polymerization. The fibrin clot caused by the sealant degrades physiologically. Fibrin sealant can be manufactured from pooled or single source donors.

The composition of fibrin glue products varies, but they generally include a 2-vial system containing fibrinogen, thrombin, factor XIII, and calcium (typically calcium chloride). Fibrin glue products generally include a first component including fibrinogen and Factor XIII (analogous to the “resin” portion of a two part epoxy kit) and a second component including thrombin in a CaCl₂ solution (analogous to the “catalyst” component of an epoxy kit). The components may be applied sequentially or simultaneously to the repair site, for example, using a double-barrel syringe onto a dry tissue bed. Prior to polymerization, the fibrin sealants acts as a flowable, sprayable “sticky” liquid that is designed to adhere to wet surfaces. Once polymerized in situ by the addition of thrombin and calcium it becomes a semi-rigid, hemostatic mass intended to hold tissue or materials in a desired configuration. Preparation takes approximately 15 minutes and once the components have been mixed, the product is available for use for 4 hours before the thrombin degrades. Used within their limitations, tissue sealants offer clinicians a valuable and versatile tool for the treatment of bleeding.

Generally, commercially available tissue sealants do not perform well in wet or “bleeding” applications. Current commercially available tissue sealants and hemostatic agents are generally either too slow, too cumbersome, lack optimum adhesive properties, or lack the tensile strength required for suturing and preventing arterial blood loss. In addition, many commercially available sealants do not have the mechanical strength to address many clinical wound closure demands.

Accordingly, improved adhesive compositions and patches are desirable.

### SUMMARY

Disclosed herein are adhesive compositions and patches, including associated systems, kits, and methods. Certain of the adhesive compositions and patches can be used to treat biological tissues (e.g., in hemostatic or other tissue treatment applications), according to certain embodiments. The subject matter of the present invention involves, in some cases, interrelated products, alternative solutions to a particular problem, and/or a plurality of different uses of one or more systems and/or articles.

In some embodiments, an adhesive composition is described. The adhesive composition comprises, according to some embodiments:
(1) a first polymeric material comprising one or more monomers of formula (m-1) cross-linked with one or more monomers of formula (m-2); and
(2) a different, second polymeric material comprising one or more monomers of formula (m-1) cross-linked with one or more monomers of formula (m-2);

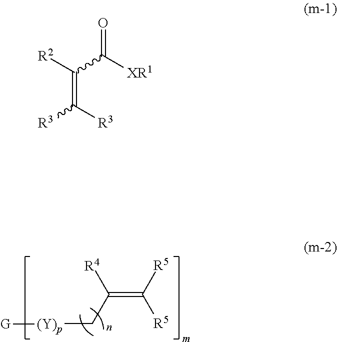

wherein:
X is O or NR¹;
each instance of R¹ is independently hydrogen or optionally substituted C₁₋₅₀alkyl;
each instance of R², R³, R⁴, and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

In some embodiments, a water-activated adhesive article is provided. The water-activated adhesive article comprises, in some embodiments, a substrate; and a water activated polymeric adhesive disposed within a solvent and in contact with the substrate.

In certain embodiments, a patch is provided. The patch comprises, according to some embodiments, a solid matrix comprising fibrin and having a moisture content of less than 20 wt %.

In some embodiments, the patch comprises a solid matrix comprising fibrin, wherein the solid matrix is substantially free of thrombin.

In certain embodiments, a method of making a patch is provided. The method comprises, in some embodiments, exposing a solid matrix comprising water and fibrin to a dehydrating agent such that water is removed from the solid matrix.

A method of applying an adhesive to a substrate is provided, in some embodiments. The method comprises, according to certain embodiments, applying an adhesive composition comprising a combination of a water-activated polymeric material and a solvent to a substrate.

According to some embodiments, a method of preparing a solid matrix comprising cross-linked fibrin is described. The method comprises, in some embodiments, applying a compressive force to a liquid containing composition comprising fibrin and/or fibrinogen; passing at least a portion of a liquid component of the composition through a filter so that at least a portion of the fibrin and/or fibrinogen is separated from the at least a portion of the liquid component; and polymerizing the fibrinogen to form fibrin and/or cross-linking the fibrin to form the solid matrix comprising cross-linked fibrin, wherein the fibrin and fibrinogen are not exposed to substantial amounts of thrombin during the preparation of the patch.

In some embodiments, a method of preparing a solid matrix comprising cross-linked fibrin comprises applying a compressive force to a liquid containing composition comprising fibrin and/or fibrinogen within a chamber; and polymerizing the fibrinogen to form fibrin and/or cross-linking the fibrin to form the solid matrix comprising cross-linked fibrin, wherein the fibrin and fibrinogen are not exposed to substantial amounts of thrombin during the preparation of the patch.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is, according to certain embodiments, a perspective view schematic illustration of a combination substrate and adhesive material;

FIG. 1B is a cross-sectional schematic diagram of a combination substrate and adhesive material, and back adhesive layer, according to some embodiments;

FIGS. 2A-2C are cross-sectional schematic illustrations of a system for producing solid matrix materials, according to one set of embodiments; and

FIG. 3 is a schematic illustration of an exemplary filter disc, used in association with one set of embodiments.

### DETAILED DESCRIPTION

Adhesive compositions and patches, and associated systems, kits, and methods, are generally described. Certain of the adhesive compositions and patches can be used to treat biological tissues (e.g., in hemostatic or other tissue treatment applications), according to certain embodiments.

Certain embodiments relate to inventive adhesive compositions. In certain embodiments, the adhesive composition comprises at least two (or more) different polymeric materials which, when mixed together and applied, demonstrate improved adherence (e.g., to biological tissue) compared to the application of the polymeric material alone.

Some embodiments are related to water activated polymeric adhesives and their use in combination with solvents. It has been discovered, according to certain embodiments, that when certain polymeric materials (including certain water activated polymeric adhesive materials and mixtures of such materials) are disposed within solvents during and/or prior to application to a substrate, the resulting adhesive is substantially stronger than the adhesive formed using the polymeric material alone (i.e., without the solvent). Some such embodiments relate to articles and methods in which the polymeric material (e.g., a water activated polymeric adhesive material) is disposed within a solvent and contacted with a substrate (e.g., a fibrin-containing substrate material or other type of substrate material). Such embodiments can be useful, for example, when one wishes to adhere the substrate to a water-containing surface, such as a tissue surface.

Additional embodiments relate to fibrin-containing substrates, such as fibrin-containing patches. In certain embodiments, the fibrin-containing substrate has a low liquid content (e.g., a low water content). Certain embodiments relate to fibrin-containing substrates that are substantially free of thrombin. The fibrin within the fibrin-containing substrate can be cross-linked, according to certain embodiments, to form a mechanically robust substrate (e.g., a tissue patch). Certain embodiments are related to methods of fabricating low-liquid (e.g., low-water) fibrin containing substrates (e.g., fibrin-containing patches). Such methods comprise, according to some embodiments, exposing a fibrin-containing substrate comprising water to a dehydrating agent (e.g., a dehydrating liquid) such that water is removed from the solid matrix.

The fibrin-containing patches can be used, according to certain embodiments, in combination with any of the inventive adhesive compositions described elsewhere herein.

As noted above, certain embodiments relate to inventive adhesive compositions. The adhesive compositions can be used, according to certain embodiments, with substrates (e.g., fibrin-containing substrates or other substrates), for example, to form tissue patches. It should be understood that the use of the adhesive compositions described herein is not limited to tissue patches, and the adhesives may have other uses.

In certain embodiments, the adhesive composition comprises at least two (or more, e.g., two, three, four, five, six, seven, eight, nine, or ten) different polymeric materials which, when mixed together and applied, demonstrate improved adherence (e.g., to biological tissue) compared to the application of the polymeric material alone. It has been discovered that, in certain cases, when two different polymeric materials as described below are mixed together and applied (e.g., to a biological tissue), the combination of the two materials leads to an unexpectedly large increase in adhesive behavior, relative to the adhesive behavior observed when the polymeric materials are applied separately from each other.

In certain embodiments, an adhesive composition is provided, comprising:
(1) a first polymeric material comprising one or more monomers of formula (m-1) cross-linked with one or more monomers of formula (m-2); and
(2) a different second polymeric material comprising one or more monomers of formula (m-1) cross-linked with one or more monomers of formula (m-2);

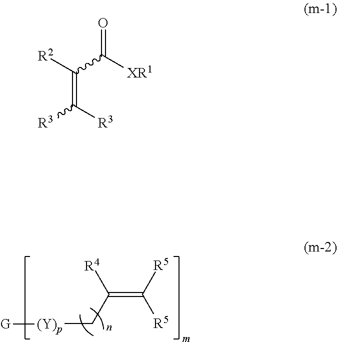

wherein:
X is O or NR¹;
each instance of R¹ is independently hydrogen or optionally substituted C₁₋₅₀alkyl;
each instance of R², R³, R⁴, and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
is 0 or 1;
n is 0 or 1, provided when p is 1, then n is 1; and
m is an integer of 2 or greater.

It is generally understood that the first polymeric material and the second polymeric material are different polymeric materials, e.g., comprise different components within their respective polymeric backbones. In certain embodiments, the first and second polymeric materials are each chemically synthesized from a different monomer (m-1) and/or a different monomer (m-2). For example, in certain embodiments, the first polymeric material is a polymer comprised of at least one type of monomer of formula (m-1) and/or at least one type of monomer of formula (m-2) not present in the polymeric backbone of the second polymeric material. In certain embodiments, the first polymeric material is a polymer comprised of at least one monomer of formula (m-1) which is not present in the polymeric backbone of the second polymeric material. In certain embodiments, the first polymeric material is a polymer comprised of at least one monomer of formula (m-2) which is not present in the polymeric backbone of the second polymeric material. In certain embodiments, the first polymeric material and the second polymeric material comprise at least one monomer of formula (m-1) which is the same, but comprise at least one monomer of formula (m-2) which is different. In certain embodiments, the first polymeric material and the second polymeric material comprise at least one monomer of formula (m-2) which is the same, but comprise at least monomer of formula (m-1) which is different. In certain embodiments, the first polymeric material and the second polymeric material comprise at least one different monomer of formula (m-1) and at least one different monomer of formula (m-2).

It is also generally understood that the first polymeric material and the second polymeric material are polymers which may comprise one type of monomer (m-1) present in the polymer backbone, or may comprise 2 or more different types of monomers (m-1), e.g., two, three, or four different types of monomers (m-1), present in the polymer backbone. For example, the first polymeric material and/or the second polymeric material may comprise a mixture of the free acid or free amide (i.e., when X is O or N, and each R¹ is hydrogen), and the corresponding ester or substituted amide (i.e., when X is O or N and at least one R¹ is optionally substituted C₁₋₅₀alkyl).

Likewise, it is also generally understood that the first polymeric material and the second polymeric material are polymers which may comprise one type of monomer (m-2), present in the polymer backbone, or may comprise 2 or more different types of monomers (m-2), e.g., 2, 3, or 4 different types of monomers (m-2), present in the polymer backbone. For example, the first polymeric material and/or the second polymeric material may comprise a mixture of monomer (m-2) wherein G is a carbohydrate and monomer (m-2) wherein G is optionally substituted aliphatic, optionally substituted heteroaliphatic, optionally substituted aryl, or optionally substituted heteroaryl.

It is also generally contemplated that the first and/or second polymeric materials may further comprise additional monomeric components within the polymeric backbone. For example, the first and/or second polymeric materials may further comprise one or more different types of monomeric polymer blocks, such as monomeric polymer blocks of cellulose polymers (e.g., hydroxyethylcellulose, ethylcellulose, carboxymethylcellulose, methyl cellulose, hydroxypropylmethylcellulose (HPMC)), dextran polymers, polymaleic acid polymers, poly(acrylic acid) polymers, poly(vinylalcohol) polymers, polyvinylpyrrolidone (PVP) polymers, and polyethyleneglycol (PEG) polymers, within the polymeric backbone. See, e.g., U.S. Patent Application Publication 20120165414 describing various macromonomers which may be possible, additional, monomeric components of the first and second polymeric materials.

However, in certain embodiments, the first and/or second polymeric materials do not further comprise additional monomeric components within the polymeric backbone. For example, in certain embodiments, the first and/or second polymeric materials are a co-polymer of one type of monomer (m-1) crosslinked by one type of monomer (m-2). Scheme 1 is an exemplary, non-limiting, depiction of a possible unit of the polymeric backbone of the first and/or second polymeric material, wherein one type of monomer (m-1) is crosslinked by one type of monomer (m-2), also referred to as monomer (m-2a), when m is 2.

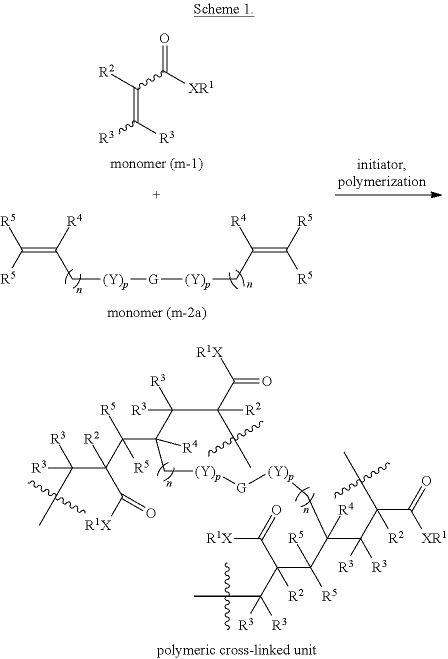

As generally described herein, the first and second polymeric material may be polymers each comprising one or more monomers of formula (m-1):

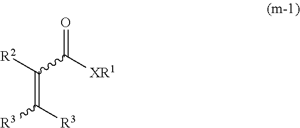

wherein X is O or NR¹; each instance of R¹ is independently hydrogen or optionally substituted C₁₋₅₀alkyl; and each instance of R² and R³ is independently hydrogen, optionally substituted C₁₋₄alkyl, or halogen.

Optional substitution may comprise, for example, hydroxyl, substituted hydroxyl, thiol, substituted thiol, amino, substituted amino, halogen, substituted or unsubstituted alkyl, substituted or unsubstituted carbocyclic, substituted or unsubstituted heterocyclic, substituted or unsubstituted aryl, and/or substituted or unsubstituted heteroaryl groups. In certain embodiments, the optional substitution comprises one or more hydroxyl groups, e.g., 1, 2, 3, 4, 5, 6, 7, 8, 9, or 10 hydroxyl groups per monomer (m-1).

In certain embodiments, X is O. In certain embodiments, X is NR¹. In certain embodiments, X is NH.

In certain embodiments, X is O and R¹ is hydrogen. In certain embodiments, X is O and R¹ is optionally substituted C₁₋₅₀alkyl, e.g., optionally substituted C₁₋₄₀alkyl, optionally substituted C₁₋₃₀alkyl, optionally substituted C₁₋₂₀alkyl, optionally substituted C₁₋₁₀alkyl, optionally substituted C₁₀₋₅₀alkyl, optionally substituted C₁₀₋₄₀alkyl, optionally substituted C₁₀₋₃₀alkyl, or optionally substituted C₁₀₋₂₀alkyl. In certain embodiments, X is O and R¹ is optionally substituted C₁₀₋₃₀alkyl.

In certain embodiments, X is NH and R¹ is hydrogen. In certain embodiments, X is NH and R¹ is optionally substituted C₁₋₅₀alkyl, e.g., optionally substituted C₁₋₄₀alkyl, optionally substituted C₁₋₃₀alkyl, optionally substituted C₁₋₂₀alkyl, optionally substituted C₁₋₁₀alkyl, optionally substituted C₁₀₋₅₀alkyl, optionally substituted C₁₀₋₄₀alkyl, optionally substituted C₁₀₋₃₀alkyl, or optionally substituted C₁₀₋₂₀alkyl. In certain embodiments, X is NH and R¹ is optionally substituted C₁₀₋₃₀alkyl.

In certain embodiments, R² is hydrogen. In certain embodiments, R² is optionally substituted C₁₋₄alkyl, e.g., optionally substituted C₁alkyl, optionally substituted C₂alkyl, optionally substituted C₃alkyl, or optionally substituted C₄alkyl. In certain embodiments, R² is —CH₃ or —CF₃. In certain embodiments, R² is halogen, e.g., fluoro.

In certain embodiments, R³ is hydrogen. In certain embodiments, R³ is optionally substituted C₁₋₄alkyl, e.g., optionally substituted C₁alkyl, optionally substituted C₂alkyl, optionally substituted C₃alkyl, or optionally substituted C₄alkyl. In certain embodiments, R³ is —CH₃ or —CF₃. In certain embodiments, R³ is halogen, e.g., fluoro.

In certain embodiments, each instance of R² and R³ is hydrogen. In certain embodiments, R² is optionally substituted C₁₋₄alkyl (e.g., —CH₃ or —CF₃) and each instance of R³ is hydrogen. In certain embodiments, R² is optionally substituted C₁₋₄alkyl (e.g., —CH₃ or —CF₃), one instance of R³ is hydrogen, and the other instance of R³ is optionally substituted C₁₋₄alkyl (e.g., —CH₃ or —CF₃). In certain embodiments, R² is hydrogen, one instance of R³ is hydrogen, and the other instance of R³ is optionally substituted C₁₋₄alkyl (e.g., —CH₃ or —CF₃).

Exemplary monomers of formula (m-1) include, but are not limited to:

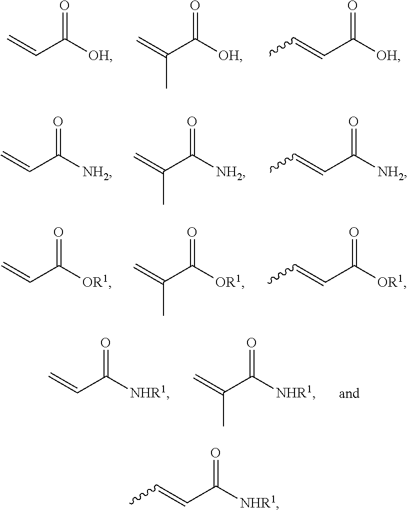

wherein R¹ is optionally substituted C₁₋₅₀alkyl, e.g., optionally substituted C₁₀₋₃₀alkyl.

In certain embodiments, the polymeric material comprises one type of monomer (m-1), e.g.:

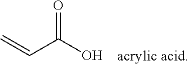

In certain embodiments, the polymeric material comprises two types of monomer (m-1), e.g., acrylic acid and a corresponding ester:

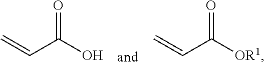

wherein R¹ is optionally substituted C₁₋₅₀alkyl, e.g., optionally substituted C₁₀₋₃₀alkyl.

Furthermore, as generally described herein, the first and second polymeric material may be polymers each comprising one or more monomers of formula (m-2):

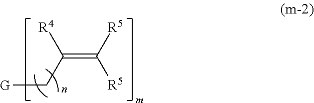

wherein each instance of R⁴ and R⁵ is independently hydrogen, optionally substituted C₁₋₄alkyl, or halogen; G is optionally substituted aliphatic, optionally substituted heteroaliphatic, optionally substituted aryl, optionally substituted heteroaryl, a polymer, or a carbohydrate; n is 0 or 1, provided when p is 1, then n is 1; and m is an integer of 2 or greater.

Optional substitution may comprise, for example, hydroxyl, substituted hydroxyl, thiol, substituted thiol, amino, substituted amino, halogen, substituted or unsubstituted alkyl, substituted or unsubstituted carbocyclic, substituted or unsubstituted heterocyclic, substituted or unsubstituted aryl, and/or substituted or unsubstituted heteroaryl groups. In certain embodiments, the optional substitution comprises one or more hydroxyl groups, e.g., 1, 2, 3, 4, 5, 6, 7, 8, 9, or 10 hydroxyl groups per monomer (m-2).

It is generally understood that G is an optionally substituted compound (i.e., an optionally substituted aliphatic, optionally substituted heteroaliphatic, optionally substituted aryl, optionally substituted heteroaryl, a polymer, or a carbohydrate group) comprising 2 or more groups of the formula (m-3) attached thereto:

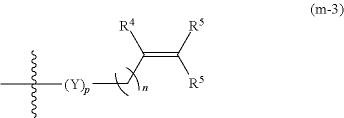

As generally defined herein, m is an integer of 2 or greater, e.g., between 2 to 1000 inclusive, between 2 to 900 inclusive, between 2 to 800 inclusive, between 2 to 700 inclusive, between 2 to 600 inclusive, between 2 to 500 inclusive, between 2 to 400 inclusive, between 2 to 300 inclusive, between 2 to 200 inclusive, between 2 to 100 inclusive, between 2 to 90 inclusive, between 2 to 80 inclusive, between 2 to 70 inclusive, between 2 to 60 inclusive, between 2 to 50 inclusive, between 2 to 40 inclusive, between 2 to 30 inclusive, between 2 to 20 inclusive, between 2 to 10 inclusive, or between 2 to 8 inclusive. It is generally contemplated when G is a polymer, m may be an integer between 2 to 1000 inclusive, as valency permits. It is further generally contemplated when G is an optionally substituted aliphatic, optionally substituted heteroaliphatic, optionally substituted aryl, optionally substituted heteroaryl, or a carbohydrate, m may be an integer between 2 to 20 inclusive, as valency permits. The phrase “or greater” and “or more” are used interchangeably herein.

In certain embodiments, m is 2, and G is an optionally substituted compound as defined herein comprising 2 groups of formula (m-3) attached thereto. In certain embodiments, m is 3, and G is an optionally substituted compound as defined herein comprising 3 groups of formula (m-3) attached thereto. In certain embodiments, m is 4, and G is an optionally substituted compound as defined herein comprising 4 groups of formula (m-3) attached thereto. In certain embodiments, m is 5, and G is an optionally substituted compound as defined herein comprising 5 groups of formula (m-3) attached thereto. In certain embodiments, m is 6, and G is an optionally substituted compound as defined herein comprising 6 groups of formula (m-3) attached thereto. In certain embodiments, m is 7, and G is an optionally substituted compound as defined herein comprising 7 groups of formula (m-3) attached thereto. In certain embodiments, m is 8, and G is an optionally substituted compound as defined herein comprising 8 groups of formula (m-3) attached thereto. In certain embodiments, m is 9, and G is an optionally substituted compound as defined herein comprising 9 groups of formula (m-3) attached thereto. In certain embodiments, m is 10, and G is an optionally substituted compound as defined herein comprising 10 groups of formula (m-3) attached thereto. In certain embodiments, m is 11, 12, 13, 14, 15, 16, 17, 18, 19, or 20, and G is an optionally substituted compound as defined herein comprising 11, 12, 13, 14, 15, 16, 17, 18, 19, or 20 groups of formula (m-3) attached thereto. In certain embodiments, m is an integer between 2 to 14, inclusive, e.g., between 2 to 12, between 2 to 10, between 2 to 8, between 2 to 6, between 4 to 12, between 4 to 10, between 4 to 18, or between 4 to 6, inclusive.

In certain embodiments, m is an integer between 20 to100, inclusive, and G is a polymer comprising 20 to 100 groups of formula (m-3) attached thereto. In certain embodiments, m is an integer between 100 to 500, inclusive, and G is a polymer as defined herein comprising 100 to 500 groups of formula (m-3) attached thereto. In certain embodiments, m is an integer between 500 to1000, inclusive, and G is a polymer comprising 500 to1000 groups of formula (m-3) attached thereto.

In certain embodiments p is 0 and Y is absent. In certain embodiments, p is 1, and Y is O. In certain embodiments, p is 1, and Y is NH.

In certain embodiments, R⁴ is hydrogen. In certain embodiments, R⁴ is optionally substituted Cᵢ₋₄alkyl, e.g., optionally substituted Cᵢalkyl, optionally substituted C₂alkyl, optionally substituted C₃alkyl, or optionally substituted C₄alkyl. In certain embodiments, R⁴ is —CH₃ or —CF₃. In certain embodiments, R⁴ is halogen, e.g., fluoro.

In certain embodiments, R⁵ is hydrogen. In certain embodiments, R⁵ is optionally substituted C₁₋₄alkyl, e.g., optionally substituted C₁alkyl, optionally substituted C₂alkyl, optionally substituted C₃alkyl, or optionally substituted C₄alkyl. In certain embodiments, R⁵ is —CH₃ or —CF₃. In certain embodiments, R⁵ is halogen, e.g., fluoro.

In certain embodiments, each instance of R⁴ and R⁵ is hydrogen. In certain embodiments, R⁴ is optionally substituted C₁₋₄alkyl (e.g., —CH₃ or —CF₃) and each instance of R⁵ is hydrogen. In certain embodiments, R⁴ is optionally substituted C₁₋₄alkyl (e.g., —CH₃ or —CF₃), one instance of R⁵ is hydrogen, and the other instance of R⁵ is optionally substituted C₁₋₄alkyl (e.g., —CH₃ or —CF₃). In certain embodiments, R⁴ is hydrogen, one instance of R⁵ is hydrogen, and the other instance of R⁵ is optionally substituted C₁₋₄alkyl (e.g., —CH₃ or —CF₃).

Exemplary monomers of formula (m-2) include, but are not limited to:

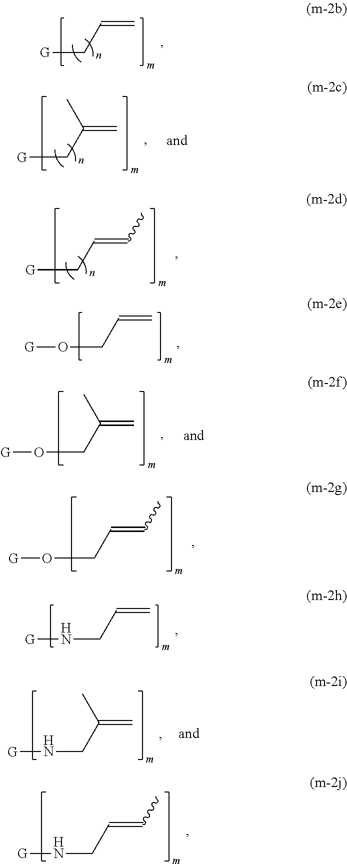

wherein n is 0 or 1, and m is an integer between 2 and 20, inclusive. In certain embodiments n is 0. In certain embodiments, n is 1. In certain embodiments, m is 2, 3, 4, 5, 6, 7, 8, 9, or 10.

In certain embodiments, G is an optionally substituted aliphatic, e.g., optionally substituted C₁₋₂₀ alkyl, optionally substituted C₂₋₂₀ alkenyl, optionally substituted C₂₋₂₀ alkynyl, or optionally substituted C₅₋₁₀carbocyclyl, comprising 2 or more groups of formula (m-3) attached thereto as valency permits, e.g., 2 to 20 groups. In certain embodiments, G is optionally substituted C₁₋₂₀ alkyl, e.g., optionally substituted C₁₋₁₀ alkyl, optionally substituted C₁₋₈ alkyl, optionally substituted C₁₋₆ alkyl, optionally substituted C₁₋₄ alkyl, optionally substituted C₂₋₁₀ alkyl, optionally substituted C₄₋₈ alkyl, or optionally substituted C₄₋₆ alkyl, comprising 2 or more groups of formula (m-3) attached thereto as valency permits, e.g., 2 to 20 groups.

In certain embodiments, G is optionally substituted C₄ alkyl, e.g., comprising 2, 3, 4, 5, 6, 7, or 8 groups of formula (m-3) attached thereto, and optionally substituted with hydroxyl groups. An exemplary monomer (m-2), wherein each of R⁴ and R⁵ is hydrogen, n is 0, m is 4, and G is C₄ alkyl substituted with hydroxyl groups, is divinyl glycol:

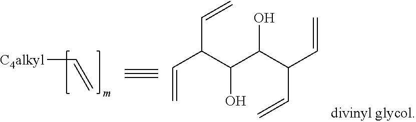

In certain embodiments, G is optionally substituted C₅ alkyl, e.g., comprising 2, 3, 4, 5, 6, 7, 8, 9, or 10 groups of formula (m-3) attached thereto, and optionally substituted with hydroxyl groups. An exemplary monomer (m-2), wherein each of R⁴ and R⁵ is hydrogen, p is 1, Y is O, n is 1, m is 4, and G is C₅ alkyl, is allyl pentaerythritol:

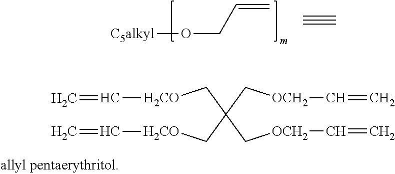

In certain embodiments, G is an optionally substituted heteroaliphatic, e.g., optionally substituted heteroC ₁₋₂₀ alkyl, or optionally substituted 5-10 membered heterocyclic, optionally substituted heteroC₂₋₂₀alkynyl, optionally substituted C₅₋₁₄ carbocyclyl, comprising 2 or more groups of formula (m-3) attached thereto as valency permits, e.g., 2 to 20 groups. In certain embodiments, G is optionally substituted heteroC₁₋₂₀ alkyl, e.g., optionally substituted heteroC₂₋₁₀ alkyl, optionally substituted heteroC₂₋₈ alkyl, optionally substituted heteroC₂₋₆ alkyl, optionally substituted heteroC₂₋₄ alkyl, optionally substituted heteroC₄₋₈ alkyl, or optionally substituted heteroC₄₋₆ alkyl, e.g., comprising 2, 3, 4, or more heteroatoms, and comprising 2 or more groups of formula (m-3) attached thereto as valency permits, e.g., 2 to 20 groups.

In certain embodiments, G is an optionally substituted aryl or optionally substituted heteroaryl comprising 2 or more groups of formula (m-3) attached thereto as valency permits, e.g., 2, 3, 4, or 5 groups, and optionally substituted with hydroxyl groups. In certain embodiments, G is an optionally substituted aryl, e.g., optionally substituted phenyl, e.g., comprising 2, 3, 4, or 5 groups of formula (m-3) attached thereto. In certain embodiments, G is an optionally substituted heteroaryl, e.g., optionally substituted 5- to 6-membered heteroaryl, e.g., comprising 2, 3, 4, or 5 groups of formula (m-3) attached thereto.

In certain embodiments, G is an polymer comprising 2 or more groups of formula (m-3) attached thereto as valency permits, e.g., 2 to 1000 groups. As used herein, a “polymer” refers to a compound comprised of at least 3 (e.g., at least 10, 20, 30, 40, 50, 60, 70, 80, 90, 100, etc.) repeating covalently bound structural units. The polymer is in certain embodiments biocompatible (i.e., non-toxic). Exemplary polymers include, but are not limited to, cellulose polymers (e.g., hydroxyethylcellulose, ethylcellulose, carboxymethylcellulose, methyl cellulose, hydroxypropylmethylcellulose (HPMC)), dextran polymers, polymaleic acid polymers, poly(acrylic acid) polymers, poly(vinylalcohol) polymers, polyvinylpyrrolidone (PVP) polymers, and polyethyleneglycol (PEG) polymers, and combinations thereof.

In certain embodiments, G is an carbohydrate comprising 2 or more groups of formula (m-3) attached thereto as valency permits, e.g., 2 to 20 groups. In certain embodiments, G is a monosaccharide comprising 2 or more groups of formula (m-3) attached thereto as valency permits, e.g., 2, 3, 4, 5, or 6 groups. In certain embodiments, G is a disaccharide comprising 2 or more groups of formula (m-3) attached thereto as valency permits, e.g., 2, 3, 4, 5, 6, 7, 8, 9, 10, 11, or 12 groups. In certain embodiments, G is a trisaccharide comprising 2 or more groups of formula (m-3) attached thereto as valency permits, e.g., 2, 3, 4, 5, 6, 7, 8, 9, 10, 11, 12, 13, 14, 15, 16, 17, 18, 19, or 20 groups. In certain embodiments, G is a disaccharide selected from the group consisting of sucrose, lactulose, lactose, maltose, trehalose, and cellobiose. An exemplary monomer (m-2), wherein each of R⁴ and R⁵ is hydrogen, p is 0 and Y is absent, n is 1, m is 8, and G is a disaccharide, is allyl sucrose:

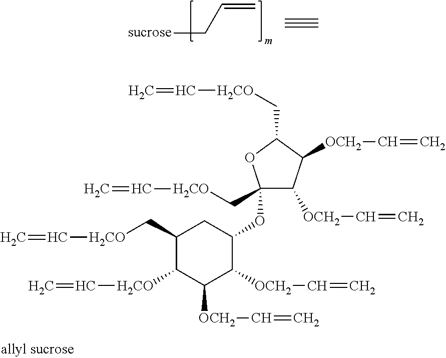

Particular combinations of monomers (m-1) and (m-2) are further contemplated herein.

For example, in certain embodiments, at least one polymeric material is a polymer of one or more monomers (m-1) cross-linked with a monomer of formula (m-2) wherein G is optionally substituted C₄ alkyl (e.g., divinyl glycol). In certain embodiments, at least one polymeric material is a polymer of one or more monomers (m-1) cross-linked with a monomer of formula (m-2) wherein G is optionally substituted C5 alkyl (e.g., allyl pentaerythritol). In certain embodiments, at least one polymeric material is a polymer of one or more monomers (m-1) cross-linked with a monomer of formula (m-2) wherein G is a carbohydrate (e.g., allyl sucrose).

In certain embodiments, at least one polymeric material is a polymer of acrylic acid and/or acrylic acid ester cross-linked with a monomer of formula (m-2) wherein G is optionally substituted C₄ alkyl (e.g., divinyl glycol). In certain embodiments, at least one polymeric material is a polymer of acrylic acid and/or acrylic acid ester cross-linked with a monomer of formula (m-2) wherein G is optionally substituted C₅ alkyl (e.g., allyl pentaerythritol). In certain embodiments, at least one polymeric material is a polymer of acrylic acid and/or acrylic acid ester cross-linked with a monomer of formula (m-2) wherein G is a carbohydrate (e.g., allyl sucrose).

In certain embodiments, at least one polymeric material is a polymer of acrylic acid cross-linked with a monomer of formula (m-2) wherein G is optionally substituted C₄ alkyl (e.g., divinyl glycol). In certain embodiments, at least one polymeric material is a polymer of acrylic acid cross-linked with a monomer of formula (m-2) wherein G is optionally substituted C₅ alkyl (e.g., allyl pentaerythritol). In certain embodiments, at least one polymeric material is a polymer of acrylic acid cross-linked with a monomer of formula (m-2) wherein G is a carbohydrate (e.g., allyl sucrose).

Commercially available polymers specifically contemplated for use herein include, but are not limited to: Carbopol® homopolymers which comprise acrylic acid crosslinked with allyl sucrose or allyl pentaerythritol (e.g., Carbopol®71G, 971P NF, 974P NF, 980 NF, 981 NF, 5984 EP, 934 NF, 934P NF, 940 NF, 941 NF);

Carbopol® copolymers which comprise acrylic acid and C₁₀₋₃₀alkyl acrylate crosslinked with allyl pentaerythritol (e.g., Carbopol®1342 NF); Carbopol® interpolymers which comprise acrylic acid and/or C₁₀₋₃₀alkyl acrylate, and a block co-polymer of polyethylene glycol and a long chain alkyl acid ester, crosslinked with allyl sucrose or allyl pentaerythritol (e.g., Carbopol® ETD2020 NF, Ultrez 10 NF);

a polycarbophil polymer, such as Noveon® AA-1 Polycarbophil, which comprises acrylic acid crosslinked with divinyl glycol;

Pemulen™ polymers which comprise acrylic acid and C₁₀₋₃₀alkyl acrylate crosslinked with allyl pentaerythritol (e.g., TR-1 NF, TR-2 NF); and

Ashland™ carbomers which comprise cross-linked polyacrylic acid (e.g., Ashland™ 940, 941, 980, and 981 carbomers).

In certain embodiments, the first and/or second polymeric materials comprise a

Carbopol® polymer, e.g., a Carbopol® homopolymer, a Carbopol® copolymer, or a Carbopol® interpolymer. In some embodiments, the first and/or second polymeric materials comprise a polycarbophil polymer, e.g. Noveon® AA-1 Polycarbophil. In some embodiments, the first polymeric material comprises a Carbopol® homopolymer, e.g., Carbopol®974P NF, while the second polymeric material comprises a polycarbophil polymer, e.g. Noveon® AA-1 Polycarbophil.

In certain embodiments, the first and/or second polymeric materials comprise carbomer homopolymers. In some embodiments, the first and/or second polymeric materials comprise polycarbophils. In some embodiments, the first polymeric material comprises a carbomer homopolymer while the second polymeric material comprises a polycarbophil.

In some embodiments, the first and/or second polymeric material can be in the form of a powder. In some such embodiments, the first and/or second polymeric material can be dissolved in a solvent prior to use (e.g., as an adhesive on a substrate, as described in more detail below).

In certain embodiments, the first and/or second polymeric materials can have a molecular weight of at least about 1000 g/mol; at least about 10,000 g/mol; at least about 100,000 g/mol; at least about 10⁶ g/mol; at least about 10⁷ g/mol; at least about 10⁸ g/mol; at least about 10⁹ g/mol; or at least about 10¹⁰ g/mol (and/or, in some embodiments, up to about 10¹⁵ g/mol, or more).

In some embodiments, the first polymeric material is biodegradable. In certain embodiments, the second polymeric material is biodegradable.

In some embodiments, the first polymeric material and the second polymeric material are present within a liquid in the adhesive composition. For example, in some embodiments, the first polymeric material and the second polymeric material are present in a solvent in which at least one of the first and second polymeric materials are soluble. Examples of solvents that can be used, alone or in combination with each other and/or other solvents described herein, include but are not limited to non-polar solvents (e.g., pentane, cyclopentane, hexane, cyclohexane, benzene, 1,4-Dioxane, chloroform, and diethyl ether); polar aprotic solvents (e.g., dichloromethane (DCM), tetrahydrofuran (THF), ethyl acetate, acetone, dimethylformamide (DMF), acetonitrile, dimethyl sulfoxide (DMSO), and propylene carbonate); polar protic solvents (e.g., formic acid, n-butanol, isopropanol, n-propanol, ethanol, methanol, acetic acid, nitromethane, water). and/or others (e.g., butylacetate, chlorobenzene, diethylether, diisoproylether, ethylmethylketone, heptane, isoamylalcohol, pentachloroethane, tetracholoethane, tetrachloromethane, toluene, and xylene). In some embodiments, the solvent comprises a non-aqueous solvent. In some embodiments, the solvent is an organic solvent. The solvent may comprise, in some embodiments, an alcohol. In some embodiments, the solvent comprises at least one of methanol, ethanol, propanol, hexane, and ethyl acetate. In certain embodiments, the solvent comprises ethanol.

In some embodiments, in the adhesive composition, the ratio of the sum of the masses of the polymeric materials (e.g., the first and second polymeric materials, and/or other polymeric materials) to the mass of the liquid in which the polymeric materials are present (e.g., the solvent) is from about 1:10 to about 10:1. In some embodiments, in the adhesive composition, the ratio of the sum of the masses of the polymeric materials to the mass of the liquid in which the polymeric materials are present is equal to or greater than 1:9, equal to or greater than 1:8, equal to or greater than 1:7, equal to or greater than 1:6, equal to or greater than 1:5, equal to or greater than 1:4, equal to or greater than 1:3, equal to or greater than 1:2, equal to or greater than 1:1.5, or equal to or greater than 1:1.2 and/or equal to or less than 9:1, equal to or less than 8:1, equal to or less than 7:1, equal to or less than 6:1, equal to or less than 5:1, equal to or less than 4:1, equal to or less than 3:1, equal to or less than 2:1, equal to or less than 1.5:1, equal to or less than 1:1, equal to or less than 0.75:1, equal to or less than 0.6:1, or equal to or less than 0.5:1.

The first polymeric material and the second polymeric material may be present in the adhesive composition in any suitable mass ratio. In some embodiments, the mass ratio of the first polymeric material to the second polymeric material within the adhesive composition is from about 1:10 to about 10:1. In some embodiments, the mass ratio of the first polymeric material to the second polymeric material within the adhesive composition is equal to or greater than 1:9, equal to or greater than 1:8, equal to or greater than 1:7, equal to or greater than 1:6, equal to or greater than 1:5, equal to or greater than 1:4, equal to or greater than 1:3, equal to or greater than 1:2, equal to or greater than 1:1.5, or equal to or greater than 1:1.2 and/or equal to or less than 9:1, equal to or less than 8:1, equal to or less than 7:1, equal to or less than 6:1, equal to or less than 5:1, equal to or less than 4:1, equal to or less than 3:1, equal to or less than 2:1, equal to or less than 1.5:1, or equal to or less than 1.2:1.

Certain embodiments are related to water activated polymeric adhesives and their use in combination with solvents. As noted above, certain embodiments relate to the discovery that when certain polymeric materials (including certain water activated polymeric adhesive materials and mixtures of such materials) are disposed within solvents during and/or prior to application to a substrate, the resulting adhesive is substantially stronger than the adhesive formed using the polymeric material alone (i.e., without the solvent). Accordingly, in some embodiments, a water-activated adhesive article is described in which a water-activated polymeric adhesive disposed within a solvent is in contact with a substrate.

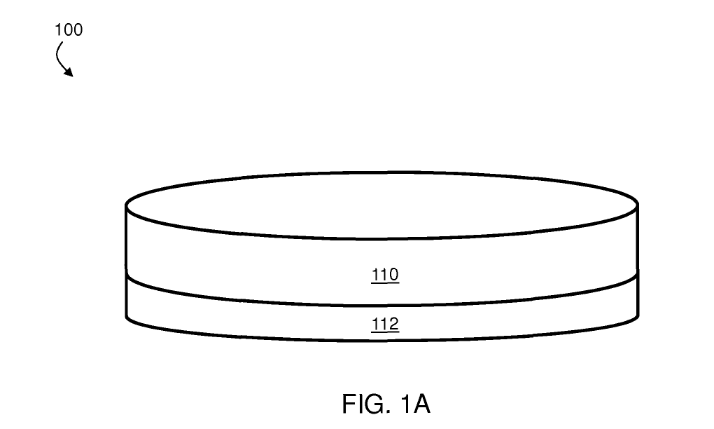

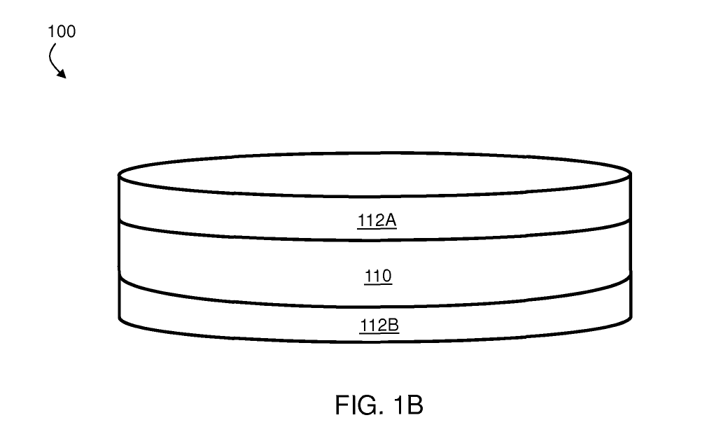

FIG. 1A is a perspective-view schematic illustration of exemplary article 100 comprising substrate 110 and an adhesive material 112 associated with substrate 110. In certain embodiments, the substrate and adhesive material can be configured to be applied to tissue such that the adhesive material contacts the tissue. In some embodiments, adhesive material 112 is substantially free of loose powder (i.e., it contains loose powder in an amount of less than about 0.1 wt %, less than about 0.01 wt %, less than about 0.001 wt %, or it contains no loose powder).

In certain embodiments, the adhesive region can help to achieve immobilization of the overlying substrate on a surface, such as a tissue surface. For example, in some embodiments, adhesive material 112 can be configured to enhance the degree to which substrate 110 is immobilized on a tissue surface onto which substrate 110 and adhesive material 112 have been applied. In some instances, immobilization of the substrate can be achieved without the need to apply much or any external pressure. In certain embodiments, immobilization of the substrate can be achieved in fewer than 5 minutes, fewer than 120 seconds, fewer than 60 seconds, or fewer than 30 seconds. In certain embodiments, once the substrate has been immobilized, it may remain in place for at least 12 hours, at least 24 hours, at least 48 hours, or at least 72 hours (and/or, in some embodiments, up to 30 days, up to 120 days, and/or until the substrate biodegrades).

In certain embodiments, the adhesive can be selected or configured such that it does not form covalent chemical bonds with the underlying surface to which it is applied (e.g., an underlying tissue surface). In certain embodiments, the adhesive region can be selected or configured to interact with the surface to which it is applied (e.g., a tissue surface) via hydrogen bonding and/or van der Waals forces. In some embodiments the adhesive material can be configured to interact with the underlying surface (e.g., tissue surface) via physisorption (sometimes also referred to as adhesive dispersion). Such adhesives can be advantageous, for example, when used to adhere tissue, at least in part because, while they effectively immobilize the patch on the tissue, they do not form strong (or permanent) bonds, which can lead to tissue damage. Of course, while non-covalently bound adhesive materials have been described, it should be understood that the invention is not limited to the use of such adhesives, and in other cases, adhesives that covalently bond to underlying surfaces (e.g., tissue surfaces) can be employed.

In some embodiments, the substrate can be applied to a tissue surface and can be allowed to integrate with the underlying tissue.

The adhesive material can be applied to or otherwise associated with the substrate via a variety of methods. For example, adhesive material 112 could be solvent cast, sprayed, brushed, or otherwise applied to solid matrix 110 or an overlying component thereof.

While FIG. 1A illustrates an embodiment in which an adhesive material is applied to one side of a substrate, in certain embodiments, adhesive material can be applied to multiple sides of the substrate. For example, in FIG. 1B, adhesive materials 112A and 112B are arranged on opposite sides of substrate 110. When arranged in this fashion, the substrate and adhesive can be used to join two surfaces, with a first surface adhering to adhesive material 112A and a second surface adhering to adhesive material 112B. For example, substrates with adhesive applied on both sides can be used to join two surfaces of skin, a pleural space, spaces between bone tissue surfaces, and other such cavities within a body.

In certain cases, and as described in more detail below, the substrate can comprise fibrin. In some such embodiments, the adhesive material is configured to immobilize the substrate (e.g., by anchoring the substrate to the tissue to which it is applied) and provide support while fibrinogen and/or fibrin from the tissue integrates with the fibrin and/or fibrinogen within the substrate. For example, fibrinogen and/or fibrin within the tissue can migrate from the tissue, through the adhesive, and into the substrate, where the fibrinogen and/or fibrin from the tissue can polymerize and/or cross-link with fibrinogen and/or fibrin within the substrate. The integration of the fibrin and/or fibrinogen within a subject's tissue with the fibrin and/or fibrinogen within the substrate can lead to the formation of a more robust interface and/or integration region between the tissue, the adhesive, and the substrate, which can produce enhanced tissue repair.

In some embodiments, the adhesive regions can be substantially free of thrombin. However, the invention is not strictly limited to thrombin-free applications, and in other embodiments, thrombin can be mixed in with and/or coated on the adhesive regions.

As illustrated in FIG. 1A, substrate 110 (which can be, for example a solid matrix) is in the form of a cylindrical disc with a substantially circular cross-sectional geometry. In other embodiments, the substrate (or the entire article, including both substrate and adhesive material) can have other cross-sectional geometries such as, for example, substantially elliptical, polygonal (e.g., including any number of sides such as in the form of a triangle, a quadrilateral (e.g., rectangular or substantially square), etc.), irregularly-shaped, or any other suitable shape.

In some embodiments, the substrate (e.g., substrate 110) and/or article (e.g., article 100) can be in the form of a sheet or film. For example, the substrate and/or article may have an aspect ratio (measured as the ratio of the maximum cross-sectional dimension to the minimum thickness of the substrate or article, for example, upon inspection) of at least about 5:1, at least about 10:1, between about 5:1 and about 100:1, or between about 5:1 and about 50:1. In certain embodiments, the substrate and/or article has an average thickness of between about 500 microns and about 1 cm. The average thickness of a component can be determined by measuring the thickness of the component at a representative number of locations and number averaging the results. In certain embodiments, the substrate and/or article has at least one cross-sectional dimension of at least about 1 cm, at least about 10 cm, at least about 50 cm, or greater. As one particular example, the substrate comprises a disc (e.g., a substantially cylindrical disc) with a thickness of between about 500 microns and about 1 cm, and a maximum cross-sectional diameter orthogonal to the thickness that is at least about 1 cm, at least about 10 cm, at least about 50 cm, or greater.

The adhesive material and the substrate can be in contact, either directly (i.e., in direct contact) or indirectly (i.e., in indirect contact), in certain embodiments. For example, as illustrated in FIG. 1A, substrate 110 and adhesive material 112 are in direct contact. However, in other embodiments, one or more solid intermediate materials can be positioned between the substrate and the adhesive material such that the substrate and the adhesive material do not contact each other directly, in which case, the substrate and the adhesive material would be said to be in indirect contact. Both articles in direct contact with each other and articles in indirect contact with each other are considered to be in contact with each other, as described herein.

In certain embodiments, adhesive material 112 comprises a water activated polymeric adhesive. Those of ordinary skill in the art are familiar with water-activated polymeric adhesives, which are adhesive polymeric materials that are rendered tacky by application of water. One can use a water-activated polymeric adhesive by applying water just prior to use, or by relying on water at the application site, to render the adhesive tacky.

Any suitable water-activated polymeric adhesive can be used. In some embodiments, the water-activated polymeric adhesive comprises any of the adhesive compositions described above that would be activated upon the application of water.

In certain embodiments, the water-activated polymeric adhesive comprises at least one (or at least two, or at least three, or more) polymeric material comprising one or more monomers of formula (m-1),

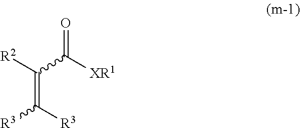

wherein:
X is O or NR¹;
each instance of R¹ is independently hydrogen or optionally substituted C₁₋₅₀alkyl; and
each instance of R² and R³ is independently hydrogen, optionally substituted C₁₋₄alkyl, or halogen.
   In some such embodiments, the water-activated polymeric adhesive comprises one or more monomers of formula (m-1) cross-linked with one of more monomers of formula (m-2),

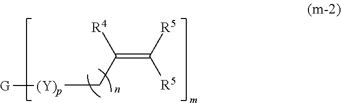

wherein:
each instance of R⁴ and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

X, R¹, R², R³, R⁴, R⁵, G, Y, p, n, and/or m can have any of the values or properties described above with respect to adhesives comprising a combination of two or more polymeric materials comprising one or more monomers of formula (m-1) cross-linked with one of more monomers of formula (m-2).

In certain embodiments, the water activated polymeric adhesive comprises a carbomer homopolymer. In some embodiments, the water activated polymeric adhesive comprises a polycarbophil. In some embodiments, the water activated polymeric adhesive comprises a carbomer homopolymer and a polycarbophil.

In certain embodiments, the water activated polymeric adhesive comprises a polymer having a molecular weight of at least about 1000 g/mol; at least about 10,000 g/mol; at least about 100,000 g/mol; at least about 10⁶ g/mol; at least about 10⁷ g/mol; at least about 10⁸ g/mol; at least about 10⁹ g/mol; or at least about 10¹⁰ g/mol (and/or, in some embodiments, up to about 10¹⁵ g/mol, or more).

In some embodiments, the water-activated polymeric adhesive comprises a polyacrylic acid. In some embodiments, the water-activated polymeric adhesive comprises a cross-linked polyacrylic acid. In some embodiments, the water-activated adhesive comprises a gum, a resin, and/or a gel. In some embodiments, the water-activated polymeric adhesive comprises a vinyl group. In certain embodiments, the water-activated polymeric adhesive comprises a co-polymer. For example, the co-polymer can be a co-polymer of a vinyl ether and maleic anhydride. In certain embodiments, the vinyl ether can comprise an alkyl vinyl ether, such that the water-activated polymeric adhesive comprises a co-polymer of an alkyl vinyl ether and maleic anhydride. The alkyl group in the alkyl vinyl ether can comprise an alkyl group containing from 1 to 18 carbons. Examples of such alkyl vinyl ethers include methyl vinyl ether, ethyl vinyl ether, propyl vinyl ether, and isobutyl vinyl ether. In certain embodiments, the vinyl ether in the co-polymer can be a divinyl ether. In certain embodiments, which can be preferred for certain applications, the water-activated polymeric adhesive comprises a co-polymer of methylvinyl ether and maleic anhydride. For example, the adhesive can comprise Gantrez MS-95.

In some embodiments, the water-activated polymeric adhesive comprising a vinyl group comprises polyvinylpyrrolidone. For example, the water-activated polymeric adhesive can comprise Kollidon®. The water-activated polymeric adhesive comprising a vinyl group can comprise, in some embodiments, a co-polymer of vinyl acetate and polyvinylpyrrolidone. For example, the water-activated polymeric adhesive can comprise, in certain embodiments, Plasdone® S-630.

Referring back to FIG. 1A, in come embodiments, adhesive material 112 comprises a water activated polymeric adhesive disposed within a solvent. In certain embodiments, at least a portion of the water-activated polymeric adhesive is soluble in the solvent. In some cases, the solvent that is used will depend upon the physical properties of the water-activated polymeric adhesive that is to be dissolved within the solvent. Examples of solvents that can be used, alone or in combination with each other and/or other solvents described herein, include but are not limited to non-polar solvents (e.g., pentane, cyclopentane, hexane, cyclohexane, benzene, 1,4-Dioxane, chloroform, and diethyl ether); polar aprotic solvents (e.g., dichloromethane (DCM), tetrahydrofuran (THF), ethyl acetate, acetone, dimethylformamide (DMF), acetonitrile, dimethyl sulfoxide (DMSO), and propylene carbonate); polar protic solvents (e.g., formic acid, n-butanol, isopropanol, n-propanol, ethanol, methanol, acetic acid, nitromethane, water). and/or others (e.g., butylacetate, chlorobenzene, diethylether, diisoproylether, ethylmethylketone, heptane, isoamylalcohol, pentachloroethane, tetracholoethane, tetrachloromethane, toluene, and xylene). In some embodiments, the solvent comprises a non-aqueous solvent. The solvent can comprise, according to certain embodiments, an organic solvent. In certain embodiments, the solvent comprises an alcohol. The solvent comprises, in some embodiments, at least one of methanol, ethanol, propanol (e.g., isopropanol), hexane, and ethyl acetate. In certain, but not necessarily all embodiments, it can be advantageous to use a solvent comprising ethanol.

Substrate 110 can be made of a number of suitable materials. The material from which substrate 110 is made can be selected based on the desired application. For example, in certain instances in which adhesive article 100 is to be adhered to tissue, substrate 110 may comprise fibrin and/or fibrinogen. Exemplary methods of making such substrates are described in more detail below.

In certain embodiments, the substrate (e.g., substrate 110 in FIG. 1A) is biodegradable. Generally, biodegradable materials are those capable of being broken down physically and/or chemically within the body of a subject (e.g., a human subject). The biodegradation may occur, for example, by hydrolysis under physiological conditions and/or by natural biological processes such as the action of enzymes present within cells or within the body, and/or by processes such as dissolution, dispersion, etc., to form smaller chemical species which can typically be metabolized and, optionally, used by the body, and/or excreted or otherwise disposed of. A polymer whose molecular weight decreases over time in vivo due to a reduction in the number of monomers is considered biodegradable. In certain embodiments, the biodegradable materials described herein (e.g., in the substrate and/or in the adhesive) can be broken down such that less than 2 wt %, less than 1 wt %, less than 0.1 wt %, or less than 0.01 wt % of their mass remains in a subject (e.g., a human subject) after fewer than 365 days, fewer than 180 days, fewer than 90 days, fewer than 60 days, or fewer than 30 days of being located within the subject.

The substrate (e.g., substrate 110 in FIG. 1A) comprises, according to certain embodiments, a polymer. For example, the substrate may comprise, in some embodiments, a biopolymer. In some embodiments, the substrate comprises a protein and/or a polysaccharide. In certain embodiments, the substrate comprises fibrin, collagen, cellulose, starch, chitosan, hyaluronic acid, polylactic acid, polyglycolic acid and/or tissue-based materials (e.g., pericardium, intestine, and/or dermal materials). The substrate can comprise, in some embodiments, a synthetic polymer. The substrate can be in the form of a paper, a cloth, a plastic sheet, or any other suitable form.

The substrate may contain a relatively low amount of liquid (e.g., water), in certain embodiments. Substrates containing low amounts of liquid, including substrates containing low amounts of water, can be particularly useful in certain embodiments in which water-activated adhesive materials are to be used with the substrate (as the low liquid content of the substrate can inhibit or prevent premature activating of the adhesive material). In some embodiments, the substrate has a liquid content of less than about 20 wt %, less than about 15 wt %, less than about 12 wt %, or less than about 10 wt %. In certain embodiments, the substrate has a water content of less than about 20 wt %, less than about 15 wt %, less than about 12 wt %, or less than about 10 wt %. In certain embodiments, the substrate is a fibrin-containing substrate having a liquid content (e.g., a water content) of less than about 20 wt %, less than about 15 wt %, less than about 12 wt %, or less than about 10 wt %. For example, any of the fibrin-containing substrates containing relatively small amounts of water, described elsewhere herein, could be used as substrate 110.

In certain embodiments, the substrate is substantially free of thrombin. An article (e.g., a substrate such as a fibrin-containing substrate, a patch, an adhesive material, etc.) is said to be “substantially free of thrombin” when the article contains thrombin in an amount of less than or equal to 0.0025 wt %. In some embodiments, an article that is substantially free of thrombin contains thrombin in an amount of less than or equal to 0.001 wt %, less than or equal to 0.00025 wt %, or less than or equal to 0.0001 wt %. In some embodiments, an article that is substantially free of thrombin is completely free of thrombin. In some embodiments, an article that is substantially free of thrombin is also substantially free of prothrombin (i.e., it contains prothrombin in an amount of less than or equal to 0.0025 wt %). In some embodiments, an article that is substantially free of thrombin and/or prothrombin contains prothrombin in an amount of less than or equal to 0.001 wt %, less than or equal to 0.00025 wt %, or less than or equal to 0.0001 wt %. In some embodiments, an article that is substantially free of thrombin and/or prothrombin is completely free of prothrombin.

Substrates including little or no thrombin can be, in certain cases, relatively less expensive to produce and may provide enhanced biocompatibility for certain applications, relative to substrates containing relatively large amounts of thrombin. In certain embodiments, the substrate is a fibrin-containing substrate that is substantially free of thrombin. For example, any of the fibrin-containing substrates that are substantially free of thrombin, described elsewhere herein, could be used as substrate 110.

Methods of applying adhesives to substrates (e.g., to form the article illustrated in FIG. 1A) are also provided. For example, certain embodiments comprise applying an adhesive composition comprising a combination of a water-activated polymeric material and a solvent to a substrate. In some such embodiments, the water-activated polymeric material and the solvent can be mixed, and the mixture can be subsequently applied to the substrate. In some embodiments, the water-activated polymeric material and the solvent can be applied to the substrate separately and subsequently mixed on the substrate. Any of the water-activated polymeric materials and substrate materials described above and elsewhere herein can be used in such methods.

While the present invention is not limited to the use of substrates made of any particular materials, certain embodiments relate to fibrin-containing substrates. The fibrin-containing substrates can be used, according to certain embodiments, as tissue patches (e.g., for the treatment of tissues, such as the hemostatic treatment of tissues). In some embodiments, the substrate has a combined fibrin and fibrinogen content of at least about 50 wt %, at least about 75 wt %, at least about 80 wt %, at least about 85 wt %, or at least about 90 wt %. In some embodiments, the substrate has a fibrin content of at least about 50 wt %, at least about 75 wt %, at least about 80 wt %, at least about 85 wt %, or at least about 90 wt %.

Certain embodiment are related to fibrin-containing substrates having low liquid (e.g., low water) content. For example, in some embodiments, a patch is provided comprising a solid matrix comprising fibrin and having a liquid content of less than about 20 wt %, less than about 15 wt %, less than about 12 wt %, or less than about 10 wt %. In some such embodiments, the patch can have a relatively high fibrin content. For example, in some embodiments, the patch has a fibrin content of at least about 50 wt %, at least about 75 wt %, at least about 80 wt %, at least about 85 wt %, or at least about 90 wt %. The fibrin-containing patch can be, according to certain embodiments, biodegradable. In some embodiments, the fibrin-containing patch is substantially free of thrombin.

Methods of fabricating fibrin-containing patches with relatively low liquid content are also provided. In certain embodiments, the fibrin-containing patch can be fabricated by exposing a solid matrix comprising water and fibrin to a dehydrating agent (e.g., a dehydrating liquid) such that water is displaced or otherwise removed from the solid matrix. In some such embodiments, at least a portion (or all) of the water within the solid matrix can be displaced and/or otherwise removed by the dehydrating agent, resulting in a patch with a relatively low water content.

A variety of dehydrating agents can be used in association with such methods. In some embodiments, the dehydrating agent comprises a dehydrating liquid. For example, the dehydrating agent can comprise an alcohol (e.g., n-butanol, isopropanol, n-propanol, ethanol, methanol). In some embodiments, the dehydrating agent comprises liquid ethanol. Examples of liquid dehydrating agents that can be used include, but are not limited to, non-polar liquids (e.g., pentane, cyclopentane, hexane, cyclohexane, benzene, 1,4-Dioxane, chloroform, and diethyl ether); polar aprotic liquids (e.g., dichloromethane (DCM), tetrahydrofuran (THF), ethyl acetate, acetone, dimethylformamide (DMF), acetonitrile, dimethyl sulfoxide (DMSO), and propylene carbonate); polar protic liquids (e.g., formic acid, n-butanol, isopropanol, n-propanol, ethanol, methanol, acetic acid, and nitromethane). and/or others (e.g., butylacetate, chlorobenzene, diethylether, diisoproylether, ethylmethylketone, heptane, isoamylalcohol, pentachloroethane, tetracholoethane, tetrachloromethane, toluene, and xylene). In some embodiments, the dehydrating agent comprises a solution (e.g., a salt solution). For example, the dehydrating agent can comprise a solution of calcium chloride (CaCl₂), calcium sulfate (CaSO₄), magnesium sulfate (MgSO₄), potassium carbonate (K₂CO₃), and/or sodium sulfate (Na₂SO₄).

In certain embodiments, non-liquid dehydrating agents can be employed. In some embodiments, the fibrin-containing substrate can be freeze dried (e.g., optionally after the addition of a humectant such as glycerin, as described below). In some embodiments, the fibrin-containing substrate can be gas dried (e.g., optionally after the addition of a humectant such as glycerin, as described below). In some embodiments, the fibrin-containing substrate can be heated (e.g., in an oven) to remove water from the fibrin-containing substrate (e.g., optionally after the addition of a humectant such as glycerin, as described below).

In certain embodiments, the fibrin-containing substrate can be exposed to a humectant. The humectant can, according to certain embodiments, ensure that the fibrin-containing substrate does not become too brittle for effective use. According to certain embodiments, the humectant can allow the fibrin-containing substrate to retain a liquid material such that the fibrin-containing substrate remains flexible. In some embodiments, the humectant is applied to the fibrin-containing patch at the same time as the dehydrating agent (described above). For example, in some embodiments, the humectant and the dehydrating agent can be mixed and applied as a mixture to the fibrin-containing substrate. In some embodiments, the humectant can be applied to the substrate before the dehydrating agent is applied to the substrate. A variety of humectants can be used. For example, in some embodiments, the humectant comprises at least one of propylene glycol, hexylene glycol, butylene glycol, glyceryl triacetate, neoagarobiose, a sugar alcohol, a polymeric polyol, quillaia, urea, aloe vera gel, MP diol, an alpha hydroxy acid, honey, and lithium. In certain embodiments, the humectant comprises at least one of glycerol, sorbitol, xylitol, and maltitol. The humectant comprises, in some embodiments, polydextrose. The humectant can comprise, in some embodiments, lactic acid. According to certain embodiments, the humectant comprises an alcohol. The alcohol can be, for example, a sugar alcohol. In some embodiments, the humectant comprises glycerol.

In certain embodiments, the dehydrating agent and the humectant can be applied as a mixture of alcohols. For example, the dehydrating agent and the humectant comprises, in some embodiments, a mixture of glycerol and a second alcohol, such as a mixture of glycerol and ethanol. According to certain embodiments, the combination dehydrating agent and humectant contains less than about 5 wt %, less than about 2 wt %, or less than about 1 wt % water. In some embodiments, the combination dehydrating agent and humectant does not contain any water. In some embodiments, the combination of the dehydrating agent and humectant contains an alcohol in an amount of at least about 5 wt %. In certain embodiments, the combination of the dehydrating agent and the humectant contains glycerol in an amount of at least about 5 wt %.

In certain embodiments, the fibrin-containing patch contains a relatively large amount of water prior to exposing the fibrin-containing patch to the dehydrating agent. For example, in some embodiments, prior to exposing the solid matrix to the dehydrating agent, the solid matrix has a water content of at least about 50 wt %, at least about 75 wt %, at least about 85 wt %, at least about 90 wt %, or at least about 95 wt % water. Exemplary methods of fabricating such patches are described in more detail below.

In some embodiments, the fibrin-containing patch to which the dehydrating agent and/or humectant is exposed can be fabricated by applying a compressive force to liquid composition comprising fibrin and/or fibrinogen, as described in more detail below.

According to some embodiments, the fibrin-containing patch comprises a relatively low amount of water after it has been exposed to the dehydrating agent. For example, in some embodiments, after exposing the solid matrix to the dehydrating agent, the solid matrix has a water content of less than about 20 wt %, less than about 15 wt %, less than about 12 wt %, less than about 10 wt %, less than about 5 wt %, less than about 2 wt %, or less than about 1 wt %.

In certain embodiments, the fibrin-containing patch comprises a relatively low amount of liquid after it has been exposed to the dehydrating agent. For example, in some embodiments, after exposing the solid matrix to the dehydrating agent, the solid matrix has a liquid content of less than about 20 wt %, less than about 15 wt %, less than about 12 wt %, or less than about 10 wt %.

In certain embodiments, an adhesive material can be disposed over the low-liquid fibrin-containing patch. The adhesive material can comprise any of the adhesive material described elsewhere herein. For example, in some embodiments, the adhesive material comprises any of the water-activated polymeric adhesives described elsewhere herein. In some embodiments, the adhesive material comprises at least one (or at least two, or at least three, or more) polymeric material comprising one or more monomers of formula (m-1), optionally cross-linked with one of more monomers of formula (m-2), as described above, including any of the substituents described above. In certain embodiments, the adhesive material disposed over the low-liquid fibrin-containing patch is substantially free of thrombin. In certain embodiments, the adhesive material disposed over the low-liquid fibrin-containing patch comprises a carbomer homopolymer. In some embodiments, the adhesive material disposed over the low-liquid fibrin-containing patch comprises a polycarbophil. In some embodiments, the adhesive material disposed over the low-liquid fibrin-containing patch comprises a carbomer homopolymer and a polycarbophil.

Certain embodiments are related to fibrin-containing substrates that are substantially free of thrombin. In certain embodiments, the patch can be substantially free of thrombin while having a relatively high fibrin and/or fibrinogen content. For example, in some embodiments, the patch is substantially free of thrombin and has a combined fibrin and fibrinogen content of at least about 50 wt %, at least about 75 wt %, at least about 80 wt %, at least about 85 wt %, or at least about 90 wt %. In some embodiments, the patch is substantially free of thrombin and has a fibrin content of at least about 50 wt %, at least about 75 wt %, at least about 80 wt %, at least about 85 wt %, or at least about 90 wt %.

In certain embodiments, the fibrin-containing patch that is substantially free of thrombin also has a relatively low liquid (e.g., water) content. For example, in some embodiments, the fibrin-containing patch that is substantially free of thrombin has a liquid content of less than about 20 wt %, less than about 15 wt %, less than about 12 wt %, or less than about 10 wt %. In certain embodiments, the fibrin-containing patch that is substantially free of thrombin has a water content of less than about 20 wt %, less than about 15 wt %, less than about 12 wt %, or less than about 10 wt %.

In certain embodiments, an adhesive material can be disposed over the fibrin-containing patch that is substantially free of thrombin. For example, any of the fibrin-containing substrates that are substantially free of thrombin (optionally also containing relatively small amounts of liquid (e.g., water)) could be used as substrate 110 in FIG. 1A. The adhesive material can comprise any of the adhesive material described elsewhere herein. For example, in some embodiments, the adhesive material comprises any of the water-activated polymeric adhesives described elsewhere herein. In some embodiments, the adhesive material comprises at least one (or at least two, or at least three, or more) polymeric material comprising one or more monomers of formula (m-1), optionally cross-linked with one of more monomers of formula (m-2), as described above, including any of the substituents described above. In certain embodiments, the adhesive material disposed over the substantially thrombin-free fibrin-containing patch is also substantially free of thrombin.

Methods of fabricating fibrin-containing substrates (e.g., patches) that are substantially free of thrombin are also provided. In certain embodiments, fibrin-containing substrates (e.g., patches) that are substantially free of thrombin can be fabricated by using non-thrombin initiators to polymerize and/or cure fibrinogen and/or fibrin when fabricating the fibrin-containing patch. In some such embodiments, the fibrin and/or fibrinogen used to form the fibrin-containing substrate (e.g., patch) are not exposed to substantial amounts of thrombin during preparation of the substrate. A material (e.g., fibrin and/or fibrinogen within a liquid medium, or any other material) is considered to be “exposed to substantial amounts of thrombin” when the material is present within a medium that contains thrombin in an amount of greater than 0.0025 wt %. In some embodiments, a material that is not exposed to substantial amounts of thrombin during a particular period of time is never present within a medium that contains thrombin in an amount of greater than 0.001 wt %, greater than 0.00025 wt %, or greater than 0.0001 wt % during that period of time. In some embodiments, a material that is not exposed to substantial amounts of thrombin over a particular period of time is never present within a medium that contains any thrombin within that period of time. In some embodiments, a material that is not exposed to substantial amounts of thrombin is also not exposed to substantial amounts of prothrombin. A material (e.g., fibrin and/or fibrinogen within a liquid medium, or any other material) is considered to be “exposed to substantial amounts of prothrombin” when the material is present within a medium that contains prothrombin in an amount of greater than 0.0025 wt %. In some embodiments, a material that is not exposed to substantial amounts of prothrombin during a particular period of time is never present within a medium that contains prothrombin in an amount of greater than 0.001 wt %, greater than 0.00025 wt %, or greater than 0.0001 wt % during that period of time. In some embodiments, a material that is not exposed to substantial amounts of prothrombin over a particular period of time is never present within a medium that contains any prothrombin within that period of time.

Exemplary methods of fabricating patches that are substantially thrombin free are described in more detail below.

Fibrin-containing substrates (e.g., patches) can be manufactured using a variety of suitable methods. In some embodiments, fibrin-containing substrates (e.g., patches) are made by applying a compressive force to a liquid-containing composition comprising fibrinogen (and/or fibrin) between two surfaces (e.g., within a syringe or other chamber). A filter can be placed within or near the volume in which the compressive force is applied to the liquid-containing composition such that unwanted material (e.g., some liquid components (e.g., water), blood cells, etc.) is passed through the filter while desirable components (e.g., fibrin and/or fibrinogen) are retained by the filter to form the fibrin-containing substrate. In this way, the concentration of fibrin (and/or fibrinogen) can be increased, potentially substantially, as the compressive force is applied to the liquid-containing composition. In addition, in some embodiments, at least a portion of the fibrinogen and/or fibrin can chemically react (e.g., the fibrinogen can polymerize to form fibrin and/or the fibrin can cross-link) before, during, and/or after application of the compressive force. Reaction and concentration via application of the compressive force (e.g., by removing at least a portion of the non-fibrin and/or non-fibrinogen components, such as liquid components (e.g., water), blood cells, and the like) can lead to the formation of a highly-concentrated, mechanically robust substrate (e.g., patch) that can be handled relatively easily and provide good structural reinforcement at a wet site, such as a bleeding wound. In certain embodiments, additional advantage, economy, convenience, and/or safety is gained by the use of autologous whole blood as the liquid-containing composition to which a compressive force is applied to form the substrate (e.g., patch).

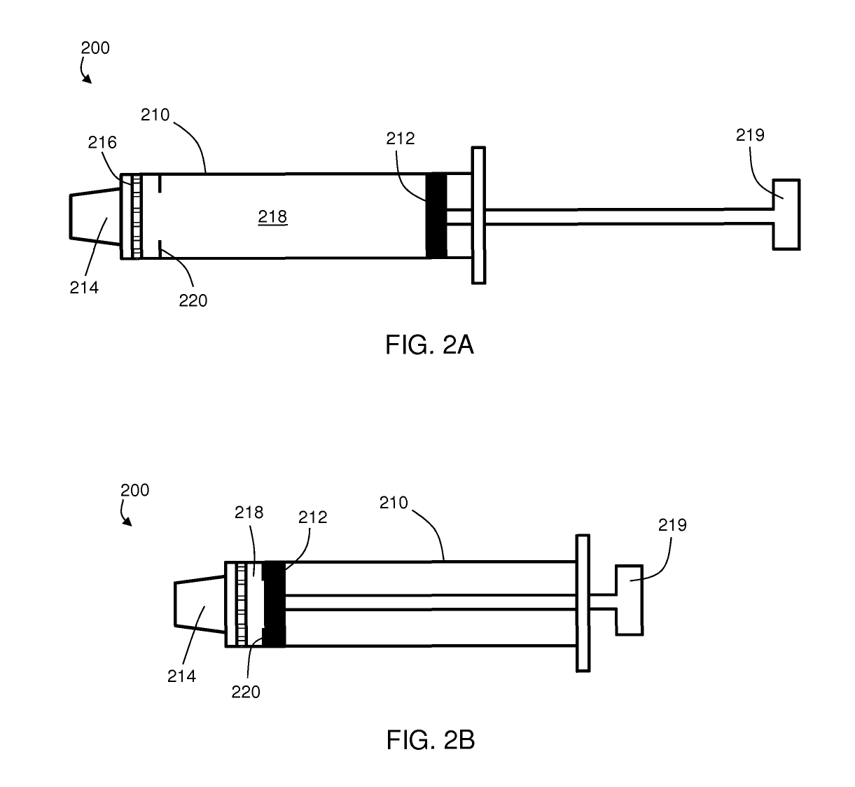

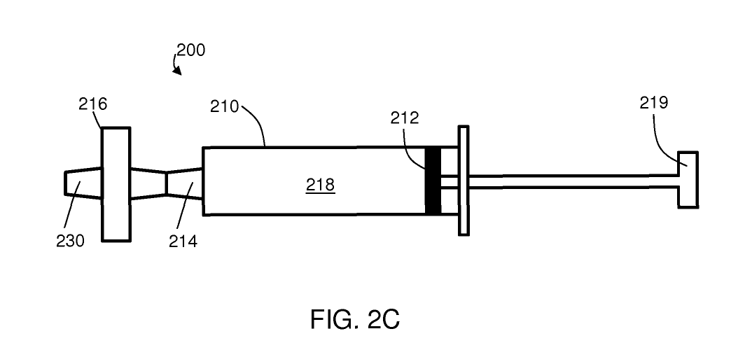

FIGS. 2A-2B are exemplary schematic illustrations outlining a system and method for the formation of a fibrin-containing substrate (e.g., patch), according to one set of embodiments. In FIGS. 2A-2B, syringe 200 comprises chamber 210. A liquid-containing composition comprising fibrin and/or fibrinogen (e.g., blood or a non-blood fibrin and/or fibrinogen suspension) can be transported into and/or provided within chamber 210. The fibrin and/or fibrinogen within the liquid-containing composition can be capable of reacting (e.g., polymerizing and/or cross-linking) within chamber 210 to form a mechanically-stable substrate (e.g., patch) material. Chemical reaction of the fibrin and/or fibrinogen can be initiated, in certain embodiments, for example, by including a curing agent within chamber 210. In some embodiments, the curing agent comprises a calcium-containing compound, such as CaCl₂. In some embodiments, chemical reaction of the fibrin and/or fibrinogen can be initiated without exposing the fibrin or fibrinogen to substantial amounts of thrombin. The invention is not limited to thrombin-free curing, however, and in other embodiments, the curing agent used to initiate chemical reaction of the fibrin and/or fibrinogen comprises thrombin.

In certain embodiments, the filter can be provided at or near a discharge end of the chamber. For example, in FIG. 2A, filter 216 is provided at or near outlet 214 of chamber 210 (within or outside chamber 210). Filter 216 can be configured to inhibit or essentially prevent the through-flow of components that are useful in forming the fibrin-containing substrate (e.g., fibrin and/or fibrinogen, and/or other useful materials), thereby retaining the useful components at or near the filter and within the chamber. In addition, filter 216 can be configured to allow at least a portion of the components of the liquid-containing composition that are not useful for forming the fibrin-containing substrate (e.g., liquid components (e.g., water), blood cells, or other similar components) to be passed through the filter and out of the chamber during the application of the compressive force (described below). Chamber 210 and filter 216 can assume a variety of geometries and can be made of a variety of materials, as described in more detail below.

In some embodiments, the fibrin-containing substrate can be formed by applying a compressive force to the liquid-containing composition within chamber 210, for example, by actuating movable wall 212 toward outlet 214. In FIG. 2A, for example, the volume 218 occupied by the liquid-containing composition is relatively large because wall 212 has not yet been actuated toward outlet 214. In FIG. 2B, on the other hand, a compressive force has been applied to chamber 210 by moving wall 212 toward outlet 214, thereby passing at least a portion of the liquid-containing composition (e.g., liquid components (e.g., water), blood cells, etc.) through filter 216 and out of chamber 210, and reducing the volume 218 of liquid-containing composition within chamber 210 and concentrating the fibrin, fibrinogen, and/or other substrate-forming components within the liquid-containing composition.

Wall 212 can be actuated using any suitable mechanism. For example, in certain embodiments, wall 212 can be actuated by manually applying a force to stopper 219. In other embodiments, wall 212 can be actuated using a trigger mechanism.

Other suitable compression mechanisms could also be used to apply a compressive force to the liquid-containing composition. For example, in some embodiments, a vacuum could applied to the volume in which the liquid-containing composition is disposed (e.g., such that the liquid-containing composition is drawn through a filter).

To illustrate one mode of operation, in one particular set of embodiments, a liquid-containing composition comprising fibrin and/or fibrinogen (and/or other components) as well as other components such as water, is provided to chamber 210. For example, a fibrin solution or blood can be provided to chamber 210. Chamber 210 can include an initiator (e.g., thrombin and/or a non-thrombin initiator such as a calcium-containing compound), which can initiate the polymerization of fibrinogen to fibrin and/or the cross-linking of fibrin. In certain embodiments, the polymerization and/or cross-linking of the fibrinogen and/or fibrin can produce fibrin molecules that are sufficiently large to be retained by filter 216. Wall 212 can be actuated toward outlet 214 such that at least a portion of the liquid (e.g., water) and/or other undesirable components (e.g., blood cells, if present, and/or other non-fibrin and/or non-fibrinogen components) are transported through filter 216 and out of outlet 214 while at least a portion of the fibrin and/or fibrinogen are retained by the filter to form a relatively concentrated matrix of material between wall 212 and filter 216. The matrix material can be solidified to form a fibrin-containing substrate, as described further below, in certain embodiments.

The chamber can comprise, in certain embodiments, a stop configured to prevent the moveable wall from reducing the volume of the chamber below a threshold value. For example, in FIGS. 2A-2B, chamber 210 includes stop 220. Stop 220 can be configured to restrict wall 212 from reducing the volume of the liquid-containing composition below the amount illustrated in FIG. 2B. Stop 220 can also be configured to restrict wall 212 from making contact with filter 216. By configuring chamber 210 and stop 220 in this way, one can reduce or eliminate the risk of applying the compressive force to the liquid-containing composition to an excessive or insufficient degree, which can help one control the final thickness of the patch.

In certain embodiments, rather than locating filter 216 within chamber 210, filter 216 can be positioned outside the chamber. For example, FIG. 2C is a cross-sectional schematic illustration of one set of embodiments in which filter 216 is fluidically connected to outlet 214 of syringe 200. In one particular set of embodiments, syringe 200 can comprise a standard syringe with a Leur-lok outlet port, and filter 216 can comprise a standard syringe disc filter cartridge. Filter 216 can also include, in some embodiments, an outlet port 230, which can be configured to allow through-flow components (e.g., water, blood cells, etc.) to be transported out of the system.

In certain embodiments, at least a portion of the fibrin and/or fibrinogen within the liquid-containing composition can chemically react (e.g., polymerize and/or cross-link) within chamber 210. Chemical reaction of the fibrin and/or fibrinogen can occur before, during, and/or after application of the compressive force. In certain embodiments, at least a portion of the fibrinogen within chamber 210 can be polymerized to form fibrin, before, during, and/or after application of the compressive force. In some embodiments, at least a portion of the fibrin within chamber 210 can be further polymerized and/or cross-linked, before, during, and/or after application of the compressive force. Chemical reaction of the fibrin and/or fibrinogen can be initiated, in certain embodiments, via a curing agent such as thrombin and/or a calcium-containing compound (e.g., CaCl₂), as discussed in more detail below. In some embodiments, chemical reaction of the fibrin and/or fibrinogen can be initiated without exposing the fibrin or fibrinogen to substantial amounts of thrombin.

In some embodiments (e.g., in embodiments in which a large amount of curing agent is present), at least a portion of the chemical reaction of the fibrin and and/or fibrinogen can occur during at least a portion of the time during which the compressive force is applied. Simultaneous application of the compressive force and reaction of the fibrin and/or fibrinogen can ensure, in certain embodiments, that the liquid-containing composition retains a suitable viscosity during the application of the compressive force. For example, if the fibrin and/or fibrinogen were polymerized and/or cross-linked to a large degree (e.g., completely) prior to applying the compressive force, application of the compressive force would be difficult due to the high resistance to flow of the viscous polymerized/gelled liquid-containing composition. Simultaneous application of the compressive force and reaction can also ensure that fibrinogen and/or fibrin are not transported out of the chamber to a large degree (e.g., by polymerizing and/or cross-linking the fibrinogen and/or fibrin to form relatively large molecules before relatively short molecules have a chance to be transported out of the chamber). By inhibiting the transport of fibrinogen and/or fibrin out of the chamber, relatively large concentrations of fibrin and/or fibrinogen within the fibrin-containing substrate can be achieved.

While chamber 210 in FIGS. 2A-2C is part of a syringe, it should be understood that the invention is not so limited. The use of a syringe can be advantageous, in certain cases, because syringes are readily available, inexpensive, and relatively easy to sterilize. Of course, in other embodiments, other types of chambers may be used to form the fibrin-containing substrates described herein. In certain embodiments, the chamber is configured such that its volume may be reduced, for example, by moving a wall of the chamber. In certain embodiments, the chamber is at least partially enclosed, including a movable wall and an outlet through which material that is not useful for forming the fibrin-containing substrate is transported. In some embodiments, the chamber can be configured to include a stop, as illustrated in the syringe chamber in FIGS. 2A-2B, to control the thickness of the fibrin-containing substrate that is produced. The moveable wall of the chamber (or any other wall of the chamber, or the filter) can be shaped, in some cases to produce a fibrin-containing substrate with a desirable surface geometry. In certain embodiments, the chamber comprises a deformable bag, and a filter could be positioned at or near an outlet through which the liquid-containing composition is transported. One of ordinary skill in the art, given the present disclosure, could envision a variety of other suitable chamber configurations that could be used to produce the fibrin-containing substrates described herein.

Chambers suitable for use in the invention can be of any desired size and can have any suitable geometry. In certain embodiments, the chamber can be configured to contain, prior to application of the compression step, at least about 1 milliliter, at least about 10 milliliters, at least about 100 milliliters, at least about 1 liter, or more (and/or, in certain embodiments, less than 10 liters or less than 1 liter). The cross-sectional shape of the chamber can be substantially circular, elliptical, polygonal (e.g., including any number of sides such as in the form of a triangle, a quadrilateral (e.g., rectangular or substantially square), etc.), irregularly-shaped, or any other suitable shape.

In addition, filter 216 can assume a variety of configurations. For example, in certain embodiments, the filter comprises a membrane disc. The membrane disc can comprise, for example, a plurality of pores. The plurality of pores can be configured and sized to separate fibrin and/or fibrinogen from at least one non-fibrin and non-fibrinogen component (e.g., liquid (e.g., water), blood cells, and the like). In one set of embodiments (including some embodiments in which the liquid-containing composition from which the fibrin-containing substrate is formed comprises blood, such as the blood sample of a subject), the filter can be configured to separate a plasma component (e.g., a plasma component within blood, which might comprise fibrin and/or fibrinogen) from at least one non-plasma component (e.g., blood cells and/or other components).

The pores within filter 216 can, in certain embodiments, comprise substantially straight passageways through a bulk filter material (as opposed to tortuous pathways that might be observed, for example, in a porous sponge). That is to say, one or more of the pores within the filter can be configured to pass from one side of the filter to the other, with a substantially constant cross-sectional geometry along substantially the entire length of the hole. For example, in one set of embodiments, filter 216 comprises a track-etched membrane. The pores within the filter can have any suitable cross-sectional shape (e.g., substantially circular, substantially elliptical, substantially square, triangular, irregular).

The pores within the filter can also be of any suitable size that is capable of achieving the desired separation (i.e. a desired level of liquid removal and retention of fibrin-containing substrate forming solids). In certain embodiments, at least about 50%, at least about 75%, or at least about 90% of the pores in the filter have maximum cross-sectional dimensions, of between about 100 micrometers and about 10 millimeters, or between about 100 micrometers and about 5 millimeters, or between about 250 micrometers and 1.5 millimeters. In some embodiments, the average pore size of the pores within the filter is between about 100 micrometers and about 10 millimeters, between about 100 micrometers and about 5 millimeters, or between about 250 micrometers and 1.5 millimeters.

In certain embodiments, at least about 50%, at least about 75%, at least about 90%, at least about 95%, or at least about 99% of the total volume of the pores in the filter is made up of pores with maximum cross-sectional dimensions, of between about 100 micrometers and about 10 millimeters, or between about 100 micrometers and about 5 millimeters, or between about 250 micrometers and 1.5 millimeters. As used herein, the volume of a pore corresponds to the volume of the voice space that is defined by the pore. For example, in a filter with cylindrical pores, the volume of any given pore is determined by calculating the volume of the cylinder defined by the pore. Volumes of individual pores can be determined by submerging the filter in a liquid and measuring the volume of liquid that is displaced, before and after individual pores are filled with a material that plugs the pores. The total volume of the pores can be calculated by plugging all of the pores, submerging the plugged filter in a fluid and measuring the volume of fluid that is displaced, and comparing this measured volume to the volume of fluid that is displaced when the filter is submerged in the fluid with all of the pores unplugged. The formula for calculating the percentage of pore volume made up of pores with maximum cross-sectional dimensions of between about X and about Y, one would sum the volumes of all of the pores with maximum cross-sectional dimensions between about X and about Y, divide this sum by the total volume of the pores in the filter, and multiply by 100%.

The pores can be arranged to have any suitable density. In certain embodiments, the density of the pores within the filter can be, for example, between about 10 and 1000, between 50 and 500, or between 100 and 200 pores per square inch.

All or part of filter 216 can be formed from a variety of suitable materials. For example, in certain embodiments, filter 216 comprises a metal such as aluminum, steel (e.g., stainless steel such as surgical stainless steel), titanium, and the like. In certain embodiments, filter 216 comprises one or more polymers. Filter 216 can comprise, in some embodiments one or more ceramics (carbide ceramics, boride ceramics, etc.). Filter 216 might also comprise a mixture (e.g., alloy or composite) or two or more of these materials. In certain embodiments, the material from which the filter is fabricated can be selected to maintain its mechanical integrity during the application of the compressive force used to produce the fibrin-containing substrate.

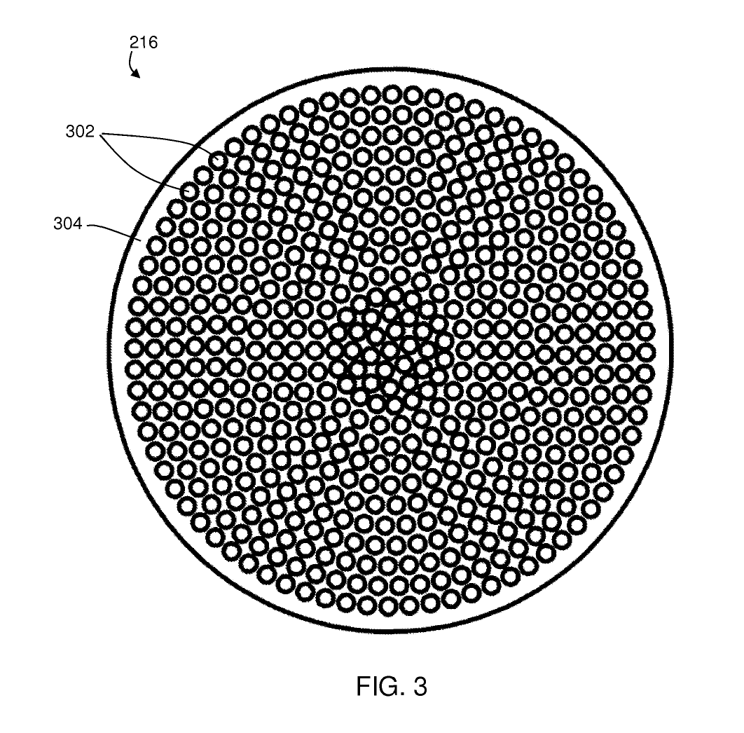

FIG. 3 is an exemplary schematic illustration of an exemplary disc filter that can be used in association with the invention, in certain embodiments. In FIG. 3, filter 216 includes a plurality of pores 302 formed in a bulk material 304.

A variety of liquid media are potentially suitable for forming the fibrin-containing substrates described herein. In certain embodiments, the liquid-containing composition used to form the fibrin-containing substrate comprises fibrin and/or fibrinogen, which can be subjected to a compressive force and/or reacted to form the fibrin-containing substrate. In some embodiments, the liquid-containing composition from which the fibrin-containing substrate is made comprises a plasma component of whole blood. The plasma component can originate from any suitable blood source. For example, the plasma component can be a plasma component of human blood, a plasma component of equine blood, a plasma component of bovine blood, and/or a plasma component of porcine blood. In some embodiments, the liquid-containing composition comprises whole blood. In certain embodiments, the liquid-containing composition can comprise a blood component, such as fibrin and/or fibrinogen or a fibrin- and/or fibrinogen-containing fraction of blood. In some embodiments, the liquid-containing composition can comprise a suspension (e.g., aqueous or non-aqueous) of fibrin and/or fibrinogen. As one particular example, in certain embodiments, the liquid-containing composition can comprise a suspension of fibrinogen formed by adding lyophilized fibrinogen to a liquid (e.g., water, saline, or any other suitable liquid) to form a fibrinogen suspension.

In certain embodiments, the liquid-containing composition supplied to the chamber (e.g., a chamber within a syringe, or any other suitable chamber) includes autologous blood. For example, in certain embodiments, the liquid-containing composition comprises at least a part of a blood sample removed from a subject. The blood sample can be transported to the chamber (e.g., directly or after separating one or more components of the blood from the remaining portion of the blood) where it can be subject to a compressive force. The fibrin and/or fibrinogen within the sample can be reacted to form a fibrin-containing substrate. In certain embodiments, the fibrin-containing substrate can be applied to the same subject from which the blood sample was removed.

Optionally, the liquid-containing composition can include (e.g., naturally or via supplementation) other components such as coagulation factors, preservatives, and/or supplemental drugs (e.g., antibiotics, anesthetics, and the like). For example, when a sample of whole blood is used as the liquid-containing composition, the sample might inherently contain coagulation factors naturally present in the blood sample. In some embodiments, a preservative might be added to the blood sample prior to its use as the liquid-containing composition. In certain embodiments in which blood is used as the liquid-containing composition, the blood can be transported essentially directly from the subject to the chamber, without chemical supplementation. In some embodiments, the liquid containing composition can include (e.g., naturally or via supplementation) one or more antimicrobial agents and/or other drugs, including those discussed in more detail below.

A curing agent can be used, in certain embodiments, to initiate polymerization, cross-linking, and/or other reactions involving the fibrin and/or fibrinogen within the liquid-containing composition. In some embodiments, the curing agent is pre-loaded into the chamber prior to adding the liquid-containing composition. The curing agent might also be added directly to the liquid-containing composition, in addition to or in place of pre-loading the chamber with the curing agent. A variety of curing agents can be employed. For example, in some embodiments, the curing agent comprises thrombin. In certain embodiments, the curing agent comprises a non-thrombin curing agent. For example, the curing agent can comprise a calcium-containing compound (e.g., compounds containing calcium ions), in place of or in addition to other curing agent components. Exemplary calcium ion-containing compounds include calcium salts such as calcium chloride (CaCl₂). In certain embodiments, the fibrinogen and/or fibrin are allowed to polymerize and/or cross-link at least partially once they have been exposed to the curing agent (e.g., thrombin, CaCl₂, etc.) prior to application of the compressive force. As noted elsewhere, in some embodiments, chemical reaction of the fibrin and/or fibrinogen can be initiated without exposing the fibrin or fibrinogen to substantial amounts of thrombin.

In some embodiments, a compressive force is applied to the liquid-containing composition, and the fibrin and/or fibrinogen are retained on a filter such that a relatively high concentration of fibrin and/or fibrinogen is present within the concentrated fibrin-containing substrate. In FIGS. 2A-2C, for example, the liquid-containing composition can be subject to a compressive force by actuating stopper 219 by hand (e.g., by employing a level of force sufficiently high to eject water or other non-substrate liquids through filter 216). In certain embodiments, after the compressive force has been applied, the sum of the concentration of the fibrin in the solid matrix and the concentration of the fibrinogen within the matrix is at least about 10, at least about 25, at least about 50, at least about 100, or between about 10 and about 150 grams per liter of the solid matrix. In some embodiments, after the compressive force has been applied, the concentration of the fibrin in the solid matrix is at least about 10, at least about 25, at least about 50, at least about 100, or between about 10 and about 150 grams per liter of the matrix.

The concentration of fibrin within the fibrin-containing substrate can be increased, in certain embodiments, by causing the fibrinogen within the liquid-containing composition to polymerize to a large degree before and/or during (and, in certain cases, after) application of the compressive force. In certain embodiments, a relatively large portion of the fibrinogen in the liquid-containing composition can be reacted to form fibrin such that the ratio of fibrin to fibrinogen in the fibrin-containing substrate is relatively high. For example, in some embodiments, the polymerization of the fibrinogen continues until a ratio of an amount of fibrin in the matrix to an amount of fibrinogen in the matrix is at least about 2:1, at least about 5:1, at least about 10:1, or at least about 100:1, by weight.

In some embodiments, the solid matrix can contain relatively highly cross-linked fibrin. Highly cross-linked fibrin can be achieved, for example, by including a cross-linking agent (e.g., thrombin, Factor XIII, and/or calcium-containing compounds, and the like) in the liquid medium to which a compressive force is applied. The degree of cross-linking can be controlled, in certain embodiments, by adjusting the amount(s) of the cross-linking agent(s) present in the liquid medium.

One of ordinary skill in the art would be capable of determining the amount of cross-linking in a given fibrin-containing medium by using one exemplary screening test in which the fibrin-containing medium is submerged in an aqueous solution of 8 molar (i.e., 8M) urea and maintained at a temperature of 25° C. Under such conditions, samples containing highly cross-linked fibrin can take a relatively long time to dissolve, while samples containing slightly cross-linked fibrin (or fibrin that is not cross-linked at all) can be dissolved relatively quickly. In certain embodiments, upon submerging the fibrin-containing substrate in an 8M aqueous solution of urea at 25° C., the fibrin-containing portion will retain its structural integrity (i.e., less than 50 wt % of the portion will dissociate) over a period of at least about 2 hours, at least about 8 hours, at least about 24 hours, at least about 48 hours, at least about 72 hours, at least about 1 week, or at least about 1 month (and/or, up to about 1 year, or longer). In certain embodiments, upon submerging the fibrin-containing substrate in a 6M aqueous solution of urea at 25° C., the fibrin-containing portion will retain its structural integrity (i.e., less than 50 wt % of the portion will dissociate) over a period of at least about 2 hours, at least about 8 hours, at least about 24 hours, at least about 48 hours, at least about 72 hours, at least about 1 week, or at least about 1 month (and/or, up to about 1 year, or longer).

Of course, the fibrin-containing substrate described herein can also be designed to include fibrin that is cross-linked to a less substantial degree, and in some cases, to include fibrin that is not cross-linked. In certain embodiments, the conditions under which the substrate is formed can be selected such that the final substrate includes the desired degree of cross-linking, for example, by adding an appropriate amount of cross-linking agent to the liquid medium to which a compressive force is to be applied.

In certain embodiments, the fibrin-containing substrate can exhibit relatively high tensile strength. Not wishing to be bound by any particular theory, the high tensile strength may result from the relatively high concentration of cross-linked fibrin in the final fibrin-containing substrate.

After the compressive force has been applied to the liquid-containing composition, a fibrin-containing solid matrix can be formed. In certain embodiments, the fibrin-containing solid matrix can be removed from the chamber in which it is formed. The fibrin-containing solid matrix can be removed from the filter while it is in the chamber, in some embodiments. In other embodiments, the filter can be removed from the chamber, after which the fibrin-containing solid matrix can be removed from the filter.

In certain embodiments, after the fibrin-containing solid matrix is removed from the chamber, the solid matrix can be exposed to a dehydrating agent (and/or a humectant), for example, as described above. The dehydrating agent can be used, for example, to remove at least a portion (or all) of the water from the solid matrix. Any of the dehydrating agents and/or humectants and/or any of the procedures for the use of such dehydrating agents and/or humectants described elsewhere herein may be used in this step.

In some embodiments, an adhesive material may be applied to the solid matrix (e.g., before or after the solid matrix is exposed to the dehydrating agent and/or humectant). Any of the adhesive materials described elsewhere herein may be applied to the solid matrix.

The fibrin-containing solid matrix can be used, for example, as a fibrin-containing substrate portion of any of the embodiments described elsewhere herein . For example, referring back to FIG. 1A, in some embodiments, the fibrin-containing solid matrix can be used as substrate 110 in article 100. As illustrated in FIG. 1A, solid matrix 110 is in the form of a cylindrical disc with a substantially circular cross-sectional geometry. In other embodiments, the solid matrix (or the entire tissue patch) can have other cross-sectional geometries such as, for example, substantially elliptical, polygonal (e.g., including any number of sides such as in the form of a triangle, a quadrilateral (e.g., rectangular or substantially square), etc.), irregularly-shaped, or any other suitable shape. The cross-sectional shape of the solid matrix and/or tissue patch can correspond to the cross-sectional shape of the chamber in which it is formed, in certain embodiments. In other embodiments, the solid matrix can be cut or otherwise shaped to assume a geometry that is different than the cross-sectional shape of the chamber in which it is formed.

In certain embodiments, the fibrin-containing solid matrix can be unsupported. Generally, unsupported solid matrix materials are those that are able to substantially retain their shape outside a container without the use of a reinforcement structure (e.g., a mesh or other reinforcement structure) within the volume of the solid matrix material. Such materials can also be referred to as self-supporting materials.

In certain embodiments, the substrates (e.g., fibrin-containing substrates), the adhesive materials, and/or the substrate/adhesive material combinations described herein can have relatively high tensile strengths. In some embodiments, the substrate, the adhesive, and/or the substrate/adhesive combination has a tensile strength of at least about 175 kPa, at least about 250 kPa, at least about 500 kPa, at least about 600 kPa, or between about 175 kPa and about 650 kPa, when measured as a true stress at break.

In some embodiments, the tissue patches described herein can be sterilized. For example, the tissue patches can be sterilized using gamma radiation. In certain embodiments, the substrate component (e.g., a fibrin-containing substrate component) can maintain its strength and/or flexibility after sterilization. For example, in some embodiments, the substrate material (e.g., material 110 in FIGS. 1A-1D) has a Young's modulus of about 10 GPa or less, of about 1 GPa or less, or of about 100 kPa or less after sterilization using gamma radiation at an intensity of 30 kGy. In some embodiments, the substrate material has a Young's modulus of from about 1 kPa to about 10 GPa, of from about 1 kPa to about 1 GPa, or of from about 1 kPa to about 100 kPa after sterilization using gamma radiation at an intensity of 30 kGy.

In some embodiments, a pharmaceutically active composition, growth factor, or other bioactive composition can be applied to a surface of and/or included within the bulk of one or more regions of any of the articles described herein (e.g., substrate 110 and/or adhesive material(s) 112 in FIG. 1A). In certain embodiments, one or more pharmaceutically active compositions can be included within and/or on a surface of the articles described herein. In some such embodiments, the article can act as a delivery mechanism for the pharmaceutically active composition. Exemplary pharmaceutically active compositions that be used in association with the articles described herein include, but are not limited to, analgesics, antimicrobial agents (e.g., antibiotics, antifungal, and/or antiviral agents), hormones, insulin, vitamins, and the like. In certain embodiments, the pharmaceutically active composition comprises a small molecule (i.e., a molecule with a molecular weight of less than about 2000 g/mole and, in some instances, less than about 1000 g/mole or less than about 500 g/mole). Exemplary small molecules include, for example, nucleic acids, peptides, polypeptides, peptide nucleic acids, peptidomimetics, carbohydrates, lipids or other organic (carbon containing) or inorganic molecules. In certain embodiments, the pharmaceutically active composition is selected from “Approved Drug Products with Therapeutic Equivalence and Evaluations,” published by the United States Food and Drug Administration (F.D.A.) (the “ Orange Book”).

In certain embodiments, an antimicrobial agent can be applied to a surface of and/or included within the bulk of one or more regions of any of the articles described herein (e.g., substrate 110 and/or adhesive material(s) 112 in FIG. 1A). The use of antimicrobial agents or other drugs can be advantageous for a variety of reasons. For example, a growing concern with the use of certain tissue sealants is that the tissue sealant can capture or contain bacteria within or under the surface of the tissue sealant and create an environment in which bacteria can grow. Including an antimicrobial agent within one or more surfaces or volumes of the article can help to combat the growth of bacteria on or around the site to which the article is applied.

A variety of antimicrobial agents can be incorporated into any of the articles described herein (e.g., substrate 110 and/or adhesive material(s) 112 in FIG. 1A). The antimicrobial agent may be bacteriocidal, virucidal, fungicidal, and/or any combination thereof. In certain embodiments, a zinc-containing material such as a zinc oxide can be used as an antimicrobial agent. Examples of suitable antimicrobial agents that can be used include, but are not limited to, metal-containing compounds (e.g., zinc-containing compounds, silver-containing compounds (e.g., silver nitrate, silver sulfadiazine, silver foams, flammacerium, Acticoat 7, Aquacel-Ag, Silvercel, and/or silver amniotic membrane), gold-containing compounds, copper-containing compounds, tin-containing compounds, chromium-containing compounds, and the like), organic antimicrobial compounds (e.g., organic antibiotics such as tetracycline antibiotics, rifampin, minocycline, and the like), antimicrobial peptide(s) (e.g., defnsins, histone H1.2, cecropin B, recombinant bactericidal/permeability-increasing protein (rBPI), and/or ceragenins), chitosan, topical antibiotics (e.g., mafenide acetate, bacitracin, mupirocin, Neosporin®, polymyxin B, nitrofurazone, and/or nystatin), iodine-based compounds (e.g., povidone-iodine, cadexomer iodine, liposomal iodine, and/or Repithel®, and/or Iocide™), and the like. Other agents that can be added to the tissue patches described herein include chlorhexidine, superoxidized water, acidified nitrite, p38MAPK inhibitor, probiotic Lactobacillus, honey, essential oils, and/or papaya.

In some embodiments, one or more growth factors can be included in and/or on a surface of any of the articles described herein (e.g., substrate 110 and/or adhesive material(s) 112 in FIG. 1A). Such growth factors can contribute to hemostasis, tissue healing, or other biological processes. For example, in certain embodiments, Platelet Derived Growth Factor (PDGF) can be included within and/or on a surface of an article (e.g., in or on substrate 110 and/or in or on adhesive material(s) 112 in FIG. 1A), which can assist in wound healing. Other examples of growth factors that be included include, but are not limited to, growth factors from one or more of the following families: adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), migration-stimulating factor, myostatin (GDF-8), nerve growth factor (NGF) and other neurotrophins, thrombopoietin (TPO), transforming growth factor alpha (TGF-α), transforming growth factor beta(TGF-β, tumor necrosis factor-alpha(TNF-α), vascular endothelial growth factor (VEGF), placental growth factor (PlGF), and the like.

In certain embodiments, a backing layer can be applied to any of the articles described herein (e.g., substrate 110 and/or adhesive material(s) 112 in FIG. 1A). However, it should be understood that backing layers are not required, and in some but not necessarily all embodiments can be advantageous to omit.

As noted elsewhere, certain of the articles described herein (e.g., adhesives, substrates, or combinations of the two) can be used as tissue patches. In certain embodiments, a tissue patch can be assembled and/or used as follows. A solid matrix (which can serve as the substrate) can be formed by applying a compressive force to a solution containing fibrin and/or fibrinogen within a container such as a syringe. In certain embodiments, the solid matrix can be removed from the syringe. An adhesive material can be placed on the solid matrix (e.g., in a thickness of about 1 millimeter). In some embodiments, the assembled patch can be applied to a tissue surface (e.g., such that the adhesive material contacts the tissue surface).

Once applied to a tissue site, blood from the subject can naturally start the coagulation process. In some embodiments, the adhesive material can provide an adhesive anchor material that holds the patch in place over the tissue, even when it is bleeding.

One advantage of the procedures outlined herein is that they can be used to quickly and easily produce tissue patches, such as fibrin-containing tissue patches (but also including other types of tissue patches). In certain embodiments, for example, the liquid-containing composition and initiator can be allowed to mix for a short period of time (e.g., in some cases for as little as 30 seconds). The step of applying a compressive force can be completed on the order of minutes (and in some cases, in as little as 30 seconds or shorter). In certain embodiments, as soon as the application of the compressive force is completed, the concentrated fibrin and/or fibrinogen material on or near the filter can be removed and used. Accordingly, in certain embodiments, the entire patch fabrication process can be completed in as little as minutes (and in certain cases, in less than 1 minute). For example, in some embodiments in which autologous blood is used to fabricate the patch, the time it takes to fabricate a patch from the time a blood sample is finished being taken to the time the patch is ready for application can be less than about 5 minutes or less than about 1 minute.

The ease with which the tissue patches described herein can be produced can provide flexibility in the way the patches are used. The patches described herein can be produced and applied directly at the site of use, in certain embodiments. For example, in some embodiments, a blood sample can be taken from a subject and added to a patch fabrication system (e.g., such as syringe 200) at the site at which the blood sample was taken. A tissue patch can be produced, removed from the fabrication system, and applied to the subject from which the blood sample was taken.

Any of the articles described herein (e.g., a substrate, adhesive material(s), and/or combinations of substrates and adhesives materials) can be packaged, according to certain embodiments. For example, in some embodiments, a substrate and/or adhesive(s) may be packaged within a foil pouch or other suitable container. In some embodiments, the container within which the article is packaged is sealed. Packaging the products described herein can allow one to store them for future use. As one particular example, in some embodiments, a solid matrix can be fabricated using a liquid-containing composition (e.g., blood sample or fibrin solution) sourced from a site remote to the site of the patch production (e.g., from a blood or plasma transfusion center). The liquid-containing composition can be used to produce a patch that is subsequently sterilized and packaged (and optionally stored for days, weeks, months, or longer) for application to a subject at a location remote from the patch production location.

In certain embodiments, the article within the package is sterile (e.g., by sterilizing the article prior to packaging the article).

In certain embodiments, the articles described herein can have a relatively long shelf life. In some embodiments, the adhesives described herein can be packaged and stored at room temperature for a period of at least lmonth, at least 6 months, or at least 1 year without losing a substantial amount (i.e., 5%) of its adhesive properties. In addition, the components used to make certain of the articles described herein (e.g., substrates, adhesives, and/or patches) can have a relatively long shelf life, especially when enclosed in a sterile package.

In another aspect, the present invention is directed to a kit including one or more of the components discussed herein.

In some embodiments, the kit comprises:

(1) a first polymeric material comprising one or more monomers of formula (m-1) cross-linked with one or more monomers of formula (m-2); and

(2) a different, second polymeric material comprising one or more monomers of formula (m-1) cross-linked with one or more monomers of formula (m-2), wherein formula (m-1), formula (m-2), and their substituents are defined as described above. The first and second polymeric materials may be arranged separately or as a mixture within the kit. The first and second polymeric materials may be in solution, in suspension, or in solid (e.g., powder) form. In some embodiments, the kit further comprises a solvent, such as any of the solvents described elsewhere herein. In some embodiments, the first and/or second polymeric materials are soluble in the solvent. In some embodiments, the kit further comprises a substrate, such as a fibrin-containing substrate (e.g., any of the substrates described elsewhere herein).

In some embodiments, the kit comprises a syringe (e.g., syringe 200 in FIGS. 2A-2C). The kit can comprise, in certain embodiments, a liquid-containing composition comprising fibrin and/or fibrinogen, such as blood, a plasma component of blood, and/or a solution of fibrin and/or fibrinogen. In some embodiments, the kit comprises a filter (e.g., filter 216 in FIGS. 2A-2C). The filter can be configured, in certain embodiments, to separate fibrin and/or fibrinogen within blood (or within another liquid containing fibrin and/or fibrinogen) from at least one other component of the blood (or from at least one other component of the fibrin- and/or fibrin-containing liquid), as described above. The kit can comprise, in certain embodiments, a curing agent. The curing agent may be substantially free of thrombin, in some embodiments. The curing agent can be capable of activating the polymerization of fibrinogen to fibrin and/or capable of activating the cross-linking of fibrin, as described above. The kit can comprise, in some embodiments, an adhesive material, including, for example, any of the adhesive materials discussed herein (e.g., in association with region 112). In some embodiments, one or more components of the kit (e.g., the syringe, the filter, the curing agent, the adhesive material, and/or other components of the kit) can be sterile.

In certain embodiments, a kit is provided including a solid matrix comprising fibrin and/or fibrinogen, which can be sterile and configured for application to a tissue surface. In some embodiments, the kit also comprises an adhesive composition. The adhesive composition can include any of the ingredients described elsewhere herein. For example, the adhesive composition can comprise at least one of the water-activated polymeric adhesives described herein. In certain embodiments, the adhesive composition in the kit may be kept separate from the solid matrix substrate in the packaging of the kit such that the adhesive composition has not yet been applied to the solid matrix substrate prior to use. In other embodiments, the adhesive composition and the solid matrix substrate can be in contact in the kit.

A “kit,” as used herein, typically defines a package or an assembly including one or more of the components of the invention, and/or other components associated with the invention, for example, as previously described. A kit of the invention may, in some cases, include instructions in any form that are provided in connection with the components of the invention in such a manner that one of ordinary skill in the art would recognize that the instructions are to be associated with the components of the invention. For instance, the instructions may include instructions for the use, modification, assembly, storage, or packaging of the components. In certain embodiments, the instructions include instructions for mixing, diluting, preserving, administering, and/or preparing compositions (e.g., blood samples, fibrinogen solutions, and the like) for use in association with the components of the kit. In some cases, the instructions may also include instructions for the use of the components or associated compositions, for example, for a particular use, e.g., to assemble a substrate/adhesive combination such as a tissue patch. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

The articles (e.g., adhesives, substrates, combinations of the two, etc.) described herein can be used in a wide variety of applications including, for example, general surgery, vascular surgery, spine surgery and ophthalmologic surgery. The articles can be configured to be applied to any type of tissue including soft tissue, bone tissue, or any other type of tissue. The articles can be employed to: assist hemostasis in a bleeding area, reduce blood flow from solid organs, assist in sealing suture holes, assist in sealing anastomosis or leaks from hollow organs, assist or replace sutures in surgical procedures (particularly where suturing is difficult or impossible), produce a water-tight closure across portions of tissue (e.g., across a suture line), reinforce tissue (e.g., in reinforcing suture lines including high stress suture lines), perform of tissue approximation, replace sutures, fill dead space or other voids in tissue, and/or in vascular repair (e.g., to seal a vascular defect). In certain embodiments, certain of the articles described herein can be employed to perform gastrointestinal suture line reinforcement, in preventing the formation of seroma (e.g., after surgical procedures), for use as soft tissue (e.g., after breast cancer or other surgical procedures in which tissue may be removed), as burn dressings, and/or for combined hemostasis/sealing and drug delivery.

In some embodiments, certain of the articles described herein (e.g., adhesives, substrates, combinations of the two, etc.) can be used to treat spleen tissue, for example, to inhibit or stop bleeding or the leaking of other bodily fluids and/or to partially or completely fill void(s) in the spleen. In certain embodiments, certain of the articles described herein can be used to treat lung tissue, for example, to inhibit or stop bleeding or the leaking of other bodily fluids, to partially or completely fill void(s) in the lung, and/or to inhibit or stop the leaking of air from the internal cavity of a lung. In some embodiments, certain of the articles described herein can be used to treat the liver, for example, to inhibit or stop bleeding or the leaking of other bodily fluids from the liver and/or to partially or completely fill void(s) in the liver. In certain embodiments, certain of the articles described herein can be used to treat heart tissue, for example, to inhibit or stop bleeding or the leaking of other bodily fluids, to partially or completely fill void(s) in the heart or associated blood vessels, and/or to inhibit or stop the leaking of blood from an internal cavity of a heart. Certain of the articles described herein can also be used to treat tissues in or near the gastrointestinal tract, for example, to inhibit or stop bleeding or the leaking of other bodily fluids, to partially or completely fill void(s) in gastrointestinal tissues.

The articles described herein can have a variety of advantageous properties, in certain although not necessarily all embodiments. For example, certain embodiments of the fibrin-containing substrates described herein can be formed and applied at the site of application. In addition, the production and application process does not require the thrombin induction of clot formation on a bleeding site. Also, the fibrin concentration of some embodiments of the patch greatly exceeds the fibrin concentration that is achieved using many traditional thrombin tissue sealants, where the only fibrin in the end thrombus is what forms at the surface of the bleeding site. Also, as noted above, articles formed according to certain embodiments of the methods described herein can have relatively high tensile strengths. Moreover, some embodiments of the articles described herein are capable of adhering to a wet (e.g., bleeding) tissue surface. Also, certain embodiments of the substrates and patches described herein are capable of chemically reacting (e.g., polymerizing and/or cross-linking) with fibrin and/or fibrinogen present at the site of application (e.g., with the fibrin and/or fibrinogen within a subject's tissue).

Certain of the substrates, adhesives, and tissue patches described herein can be biocompatible and/or biodegradable. In addition, the substrates, adhesives, and/or tissue patches can be configured such that they do not interfere with any metabolic pathways that would produce significant biologic dysfunction. The use of sterile materials and components to form certain embodiments of the articles described herein can reduce or eliminate the risk of bacterial, viral, or other infectious agents being transmitted as the result of the use of the article.

The articles described herein (e.g., substrates, adhesives, tissue patches, etc.) can be used to treat human subjects, in certain embodiments. In other embodiments, the articles described herein can be used to treat non-human animal subjects. For example, in certain cases, the articles described herein can be used in veterinary applications, for example, those involving horses, dogs, cats, and the like.

The following patent publications are incorporated herein by reference in their entirety for all purposes: International Patent Application Serial No. PCT/US2013/024322 filed Feb. 1, 2013, published as International Patent Publication No. WO 2013/116633 on Aug. 8, 2013, and entitled “Tissue Patches and Associated Systems, Kits, and Methods”; U.S. patent application Ser. No. 13/644,868 filed Oct. 4, 2012, published as U.S. Patent Publication No. US 2013/0202656 on Aug. 8, 2013, and entitled “Systems and Kits for the Fabrication of Tissue Patches”; U.S. patent application Ser. No. 13/644,889 filed Oct. 4, 2012, published as U.S. Patent Publication No. US 2013/0202674 on Aug. 8, 2013, and entitled “Tissue Patch”; and U.S. patent application Ser. No. 13/644,907 filed Oct. 4, 2012, published as U.S. Patent Publication No. US 2013/0202675 on Aug. 8, 2013, and entitled “Systems and Methods for the Fabrication of Tissue Patches.”

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Compounds can be present as individual isomers substantially free of other isomers, and alternatively, as mixtures of various isomers.

Unless otherwise stated, compounds depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of this disclosure.

When a range of values is listed, it is intended to encompass each value and sub-range within the range. For example “C₁₋₆ alkyl” is intended to encompass, C₁, C₂, C₃, C4, C5, C6, C_{1-6,} C_{1-5,} C_{1-4,} C_{1-3,} C_{1-2,} C_{2-6,} C_{2-5,} C_{2-4,} C_{2-3,} C_{3-6,} C_{3-5,} C_{3-4,} C_{4-6,} C_{4-5,} and C₅₋₆ alkyl.

As used herein, “aliphatic” refers to alkyl, alkenyl, alkynyl, and carbocyclic groups, as defined herein. Likewise, the term “heteroaliphatic” as used herein, refers to heteroalkyl, heteroalkenyl, heteroalkynyl, and heterocyclic groups, as defined herein.

As used herein, “alkyl” refers to a straight—chain or branched saturated hydrocarbon group having from 1 to 50 carbon atoms (“C₁₋₅₀ alkyl”). In some embodiments, an alkyl group has 1 to 40 carbon atoms (“C₁₋₄₀ alkyl”). In some embodiments, an alkyl group has 1 to 30 carbon atoms (“C₁₋₃₀ alkyl”). In some embodiments, an alkyl group has 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₅₀ alkyl (e.g., —CH₃). In certain embodiments, the alkyl group is a substituted C₁₋₅₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 50 carbon atoms (“C₁₋₅₀ haloalkyl”). In some embodiments, the haloalkyl moiety has 1 to 40 carbon atoms (“C₁₋₄₀ haloalkyl”). In some embodiments, the haloalkyl moiety has 1 to 30 carbon atoms (“C₁₋₃₀ haloalkyl”). In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a perchloroalkyl group. Examples of haloalkyl groups include —CF ₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, 4, or more heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 50 carbon atoms and 1 or more heteroatoms within the parent chain (“heteroC₁₋₅₀ alkyl”). In some embodiments, a heteroalkyl group is a saturated group having 1 to 40 carbon atoms and 1 or more heteroatoms within the parent chain (“heteroC₁₋₄₀ alkyl”). In some embodiments, a heteroalkyl group is a saturated group having 1 to 30 carbon atoms and 1 or more heteroatoms within the parent chain (“heteroC₁₋₃₀ alkyl”). In some embodiments, a heteroalkyl group is a saturated group having 1 to 20 carbon atoms and 1 or more heteroatoms within the parent chain (“heteroC₁₋₂₀ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC_{1 2} alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC_{2 6} alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₅₀ alkyl. In certain embodiments, the heteroalkyl group is a substituted heteroC₁₋₅₀ alkyl.

As used herein, “alkenyl” refers to a straight—chain or branched hydrocarbon group having from 2 to 50 carbon atoms and one or more carbon-carbon double bonds (e.g., 1, 2, 3, 4, or more double bonds). In some embodiments, an alkenyl group has 2 to 40 carbon atoms (“C₂₋₄₀ alkenyl”). In some embodiments, an alkenyl group has 2 to 30 carbon atoms (“C₂₋₃₀ alkenyl”). In some embodiments, an alkenyl group has 2 to 20 carbon atoms (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon—carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₅₀ alkenyl. In certain embodiments, the alkenyl group is a substituted C₂₋₅₀ alkenyl.

As used herein, “heteroalkenyl” refers to an alkenyl group as defined herein which further includes at least one heteroatom (e.g., 1, 2, 3, 4, or more heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 50 carbon atoms, at least one double bond, and 1 or more heteroatoms within the parent chain (“heteroC₂₋₁₀ alkenyl”). In some embodiments, a heteroalkenyl group has 2 to 40 carbon atoms at least one double bond, and 1 or more heteroatoms within the parent chain (“heteroC₂₋₄₀ alkenyl”). In some embodiments, a heteroalkenyl group has 2 to 30 carbon atoms at least one double bond, and 1 or more heteroatoms within the parent chain (“heteroC₂₋₃₀ alkenyl”). In some embodiments, a heteroalkenyl group has 2 to 20 carbon atoms at least one double bond, and 1 or more heteroatoms within the parent chain (“heteroC₂₋₂₀ alkenyl”). In some embodiments, a heteroalkenyl group has 2 to 10 carbon atoms at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₅₀ alkenyl. In certain embodiments, the heteroalkenyl group is a substituted heteroC₂₋₅₀ alkenyl.

As used herein, “alkynyl” refers to a straight—chain or branched hydrocarbon group having from 2 to 50 carbon atoms and one or more carbon-carbon triple bonds (e.g., 1, 2, 3, 4, or more triple bonds) (“C₂₋₅₀ alkynyl”). Alkynyl may further include one or more carbon-carbon double bonds (e.g., 1, 2, 3, 4, or more double bonds). In some embodiments, an alkynyl group has 2 to 40 carbon atoms (“C₂₋₄₀ alkynyl”). In some embodiments, an alkynyl group has 2 to 30 carbon atoms (“C₂₋₃₀ alkynyl”). In some embodiments, an alkynyl group has 2 to 20 carbon atoms (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C_{2 9} alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon—carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₅₀ alkynyl. In certain embodiments, the alkynyl group is a substituted C₂₋₅₀ alkynyl.

As used herein, “heteroalkynyl” refers to an alkynyl group as defined herein which further includes at least one heteroatom (e.g., 1, 2, 3, 4, or more heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 50 carbon atoms, at least one triple bond, and 1 or more heteroatoms within the parent chain (“heteroC₂₋₄₀ alkynyl”). In some embodiments, a heteroalkynyl group has 2 to 40 carbon atoms, at least one triple bond, and 1 or more heteroatoms within the parent chain (“heteroC₂₋₄₀ alkynyl”). In some embodiments, a heteroalkynyl group has 2 to 30 carbon atoms, at least one triple bond, and 1 or more heteroatoms within the parent chain (“heteroC₂₋₃₀ alkynyl”). In some embodiments, a heteroalkynyl group has 2 to 20 carbon atoms, at least one triple bond, and 1 or more heteroatoms within the parent chain (“heteroC₂₋₂₀ alkynyl”). In some embodiments, a heteroalkynyl group has 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and for 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC_{2 6} alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₅₀ alkynyl. In certain embodiments, the heteroalkynyl group is a substituted heteroC₂₋₅₀ alkynyl.

As used herein, “carbocyclyl” or “carbocyclic” refers to a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C_{3 6} carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1] heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5] decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro-fused ring system such as a bicyclic system (“bicyclic carbocyclyl”) or tricyclic system (“tricyclic carbocyclyl”)) and can be saturated or can contain one or more carbon—carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C_{3 10} cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C_{5 6} cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, “heterocyclyl” or “heterocyclic” refers to a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro-fused ring system such as a bicyclic system (“bicyclic heterocyclyl”) or tricyclic system (“tricyclic heterocyclyl”)), and can be saturated or can contain one or more carbon—carbon double or triple bonds. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “carbohydrate” refers to a monosaccharide, a disaccharide (2 monosaccharide units bound together by a glycosidic linkage), or a trisaccharide (3 monosaccharide units bound together by glycosidic linkages). See, e.g., McMurry, John. Organic Chemistry. 7th ed. Belmont, Calif.: Thomson Brooks/Cole, 2008. Monosaccharides are the simplest carbohydrates in that they cannot be hydrolyzed to smaller carbohydrates. Most monosaccharides can be represented by the general formula C_{y}H_{2y} O_{y} (e.g., C₆H¹²O₆ (a hexose such as glucose)), wherein y is an integer equal to or greater than 3, but certain monosaccharides not represented by this general formula may also be considered monosaccharides, e.g., for example, deoxyribose is of the formula C₅H₁₀ O ₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Exemplary disaccharides include sucrose, lactulose, lactose, maltose, trehalose, and cellobiose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

As used herein, “aryl” refers to a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

As used herein, “heteroaryl” refers to a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC_{2 10} alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C_{3 10} carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC_{1 10}alkyl, heteroC_{2 10}alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C_{1 10} alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁻X⁺, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C_{1≢}perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, _{C6 10} aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆alkyl)₂, —N(C₁₋₆ alkyl)₃⁺X⁻, —NH(C_{1 6} alkyl)₂³⁰ X⁻, —NH₂(C₁₋₆ alkyl) ³⁰ X⁺, —NH₃³⁰ X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C_{1 6} alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C_{2 6} alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻is a counterion.

In certain embodiments, a carbon atom substituent is selected from the group consisting of halogen, —CN, —NO₂, —N₃, —SO₂H, —SO₃H, —OH, —OR^{aa}, —N(R^{bb})₂, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb)}₂, —C(═O)NR^{bb}SO₂R^{aa}, —NR^{bb}SO₂R^{aa}, —SO₂N (R^{bb})₂, —SO₂R^{aa}, —S(═O)R^{aa}, —Si(R^{aa})₃, C₁₋₁₀ alkyl, C₁₋₁₀ perhaloalkyl, C₂₋₁₀ alkenyl, C₂₋₁₀ alkynyl, heteroC₁₋₁₀ alkyl, heteroC_{2 10} alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F, Cl, Br, I), NO₃^{-,} ClO₄³¹ , OH⁻H₂PO₄, HSO₄⁻, sulfonate ions (e.g., methansulfonate, trifluoromethanesulfonate, ρ-toluenesulfonate, benzenesulfonate, 10-camphor sulfonate, naphthalene-2-sulfonate, naphthalene-1-sulfonic acid-5-sulfonate, ethan-l-sulfonic acid-2-sulfonate, and the like), and carboxylate ions (e.g., acetate, ethanoate, propanoate, benzoate, glycerate, lactate, tartrate, glycolate, and the like).

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

As used herein, the term, “amino” refers to the group —NH₂. The term “substituted amino,” by extension, refers to a monosubstituted amino, a disubstituted amino, or a trisubstituted amino, as defined herein. In certain embodiments, the “substituted amino” is a monosubstituted amino or a disubstituted amino group.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa} , —NR^{bb}CO₂R^{aa}, —NR^{bb}C(═O)N(R^{bb})₂, —NR^{bb}C(═NR^{bb})N(R^{bb})₂, NR^{bb}SO₂R^{aa}, —NR^{bb}P(═O)(OR^{cc})₂, and —NR^{bb} P(═O)(NR^{bb})₂, wherein R^{aa}, R^{bb}, and R^{cc} are as defined herein, with the proviso that the nitrogen atom directly attached to the parent molecule is not substituted with hydrogen.

As used herein, the term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻are as defined herein.

A “quaternary salt” refers to a nitrogen atom directly attached to or part of the parent compound or parent chain which comprises two to four substituents or groups attached thereto such that the nitrogen has a valency of four, wherein the nitrogen atom is positively charged, and the charge is balanced with a counteranion. Exemplary quaternary salts include but are not limited to a substituent amine attached to the parent compound or chain —N(R^{bb})₃⁺X⁻or an amine part of the parent chain -—N(R^{bb})₂-³⁰ X⁻, wherein R^{bb} and X⁻are as defined herein.

Nitrogen atoms can be substituted or unsubstituted as valency permits, and include primary, secondary, tertiary, and quarternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂. —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa} , R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The invention is not intended to be limited in any manner by the above exemplary listing of substituents.

### EXAMPLES

The following examples are intended to illustrate certain embodiments of the present invention, but do not exemplify the full scope of the invention.

### Example 1

The following example describes the fabrication of a fibrin-containing solid matrix, which can be used as a substrate in any of the embodiments described elsewhere herein (e.g., as a tissue patch).

Fibrin-containing solid matrices were fabricated using both whole blood and a liquid-containing composition comprising human blood plasma (Seraplex, Pasadena, Calif. (sourced from the American Red Cross)). The human plasma was brought to room temperature, and placed into a slip tip syringe. Subsequently, 2 M CaCl₂ and thrombin were added to the syringe. The syringe was incubated at 37° C. for 15 minutes.

A rigid disc filter was placed within a filter holder. For example, for the 25 mm diameter patches, a Swinnex Filter Holder (47 mm, Catalog Number SX0004700, EMD Millipore Corporation, Billerica, Mass.) was used. The square solid matrices were fabricated using custom made filters. The filter holder was attached to the discharge end of the syringe, similar to the arrangement illustrated in FIG. 1C. As one example, a disc filter similar to the filter illustrated in FIG. 3 was used to fabricate circular solid matrices. The disc filters were made by forming a plurality of 0.047-inch diameter pores in a 1.5 millimeter thick polyolefin disc.

After loading and incubation, a compressive force was applied, by hand with minimal back pressure, to the liquid-containing composition within the syringe. The curing agents rapidly induced substantially complete clot formation within the syringe. As a compressive force was applied to the liquid media within the syringe, the filter allowed substantially all of the non-gelatinous material (e.g., water) to pass across, but retained and concentrated substantially all of the gelatinous portion (which formed the solid matrix).

After the compressive force was applied to the syringe, the filter holder was removed from the syringe, and disassembled. The filter was removed from the disassembled filter holder, and the solid matrix was dislodged from the filter using a gloved finger.

Fibrin-containing solid matrices of various sizes were fabricated. For example, 25 mm diameter circular matrices were formed by loading 20 mL of plasma, 200 μL of 2M CaCl₂, and 200 μL of thrombin into a 20 mL slip tip syringe. 2.5-inch by 2.5-inch square patches were fabricated by loading 60 mL of plasma, 600 μL of 2M CaCl₂, and 600 μL of thrombin into a 60 mL slip tip syringe and passing the contents of the syringe across a square filter within a filter holder. 4-inch by 4-inch square patches were fabricated by loading 120 mL of plasma, 1.2 mL of 2M CaCl₂, and 1.2 mL of thrombin into a 140 mL slip tip syringe and passing the contents of the syringe across a square filter within a filter holder. Thrombin-free fibrin-containing solid matrices were also fabricated using the same process as outlined above, but without adding thrombin to the liquid compositions within the syringes. For the thrombin-free fibrin-containing solid matrices, the syringe was incubated at 37° C. for 12 hours, rather than the 15 minute incubation performed for thrombin-containing formulations. The thrombin-free fibrin-containing solid matrices had similar mechanical properties as those of the solid matrices containing thrombin.

### Example 2

This example describes the removal of liquid from fibrin-containing solid matrices by exposing the matrices to a dehydrating agent in combination with a humectant.

The fibrin-containing solid matrices fabricated in Example 1 typically contained water in an amount of about 80 wt %. To reduce the liquid content of the fibrin-containing solid matrices, the solid matrices fabricated as described in Example 1 were exposed to a dehydrating agent. First, the fibrin-containing solid matrix was first rinsed for 30-60 seconds in a beaker containing 0.9 wt % NaCl. Next, the fibrin-containing solid matrix was placed into a beaker containing a mixture comprising 90 vol % of a pure ethanol dehydrating agent and 10 vol % of a glycerol humectant (99.5 vol % glycerol in 0.5 vol % water). The fibrin-containing solid matrix was left in the beaker for 1-15 minutes at room temperature. The fibrin-containing solid matrix was then blotted dry with a paper towel and allowed to dry further at room temperature.

The moisture content of the resulting fibrin-containing solid matrix was determined by weighing the solid matrix before and after drying the solid matrix at 37 ° C. for 24 hours. It was found that the solid matrix, after exposure to the dehydrating agent, contained, on average, less than 8 wt % moisture (with individual samples ranging between 6.3 wt % and 9.9 wt % moisture).

### Example 3

This example describes the production of a polyacrylic acid-based adhesive and its use with the fibrin-containing solid matrices described in Examples 1 and 2.

An adhesive composition was made using a blend of Carbopol® 974P NF and Noveon® AA-1 Polycarbophil acrylic acid polymers (The Lubrizol Corporation, Wickliffe, Ohio, USA). Carbopol® 974P NF is a high molecular weight acrylic acid polymer crosslinked with allyl pentaerythritol. Noveon® AA-1 Polycarbophil is a high molecular weight acrylic acid polymer crosslinked with divinyl glycol.

A 1:1 mass ratio blend of Carbopol® 974P NF and Noveon® AA-1 Polycarbophil powders was thoroughly mixed. Pure ethanol was added to the powder mixture to form a paste, with 2 μL of pure ethanol being per milligram of mixed powder.

Immediately forming the paste, the paste was applied to the dehydrated fibrin-containing solid matrix (described in Example 2). A parafilm sheet was placed over the top of the adhesive paste, and pressure was applied to spread the adhesive paste across the surface of the fibrin-containing solid matrix. Finally, fibrin-containing substrate and the adhesive paste were allowed to dry at room temperature for 24-72 hours.

After 24 hours, the adhesive paste was extremely tacky to the touch, and was essentially transparent. For a subset of the experiments, the resulting patch (including the fibrin-based solid matrix and the adhesive) , was placed into a foil pouch and heat sealed. Patches stored in heat sealed adhesive pouches exhibited no substantial decrease in adhesion after when stored at room temperature over a period of 1 month. It is expected that the adhesive-coated tissue patches will have a shelf life of over 1 year while maintaining its adhesive properties.

Additional adhesive compositions were made using only Carbopol® 974P NF acrylic acid polymers. Pure ethanol was added to the Carbopol® 974P NF powder to form a paste, with 2 μL of pure ethanol being per milligram of powder. The resulting adhesives exhibited substantially diminished adhesive properties relative to the adhesives made using mixtures of Carbopol® 974P NF and Noveon® AA-1 Polycarbophil acrylic acid polymers.

Additional adhesive compositions were made using only Noveon® AA-1 Polycarbophil acrylic acid polymers. Pure ethanol was added to the Noveon® AA-1 Polycarbophil powder to form a paste, with 2 μL of pure ethanol being per milligram of powder. The resulting adhesives exhibited substantially diminished adhesive properties relative to the adhesives made using mixtures of Carbopol® 974P NF and Noveon® AA-1 Polycarbophil acrylic acid polymers. These adhesives also exhibited diminished adhesive properties relative to the adhesives made using only Carbopol® 974P NF acrylic acid polymers.

### Example 4

This example describes the use of the adhesive-coated solid matrices, produced using the methods described in Examples 1-3, to provide hemostasis at a spleen injury site. Bleeding from solid organs such as the spleen, liver, or kidney can occur due to external trauma or during surgical intervention and is a commonly encountered clinical indication of use of surgical hemostatic agents and/or sealants.

Tissue patches comprising fibrin-containing substrates (fabricated using the methods described in Examples 1 and 2) coated with adhesive (as described in Example 3) were evaluated via experiments on a pig following procedures outlined in Browdie, D. A., et al., “Tests of Experimental Tissue Adhesive Sealants,” Texas Heart Institute Journal, 2007, 34, pp. 313-317.

A representative injury was created by amputating the distal 1-2 cm of the tail of the spleen (most distal aspect of the organ relative to blood supply) after which brisk, mixed arterial/venous bleeding was observed. The amputation was a full thickness resection through the dorsal and ventral splenic capsule as well as the splenic parenchyma. The injury site was covered with a gauze sponge to absorb surface blood. The sponge was then removed, and an adhesive-coated tissue patch fabricated according to the methods described in Examples 1-3 was applied to the injury site. The tissue patch was of sufficient size to cover the exposed parenchyma and overlap at least 2 cm onto adjacent intact splenic capsule. The tissue patch was centered over the injury and held tightly against the spleen, with another gauze sponge behind the tissue patch, for 1 minute. The sponge was removed, leaving behind the tissue patch adhered to the tissue. Bleeding was assessed by looking for the escape of blood either from beneath the patch edges or through the body of the patch. No bleeding was observed immediately after application, nor was any bleeding observed over the ensuing 2 hours of observation.

A second injury type was created in the spleen by resecting a 10 mm circular portion of the capsule while cutting approximately 2 mm deep into the parenchyma of the organ. Similarly, the surface was sponged to remove blood, an adhesive-coated tissue patch allowing 2 cm of overlap onto adjacent capsule was applied, and pressure was held against the spleen for 1 minute using a gauze sponge. This injury was repeated at 5 distinct sites along the ventral surface of the spleen. Complete hemostasis was observed at all patch application sites.

### Example 5

This example describes the use of the adhesive-coated solid matrices, produced using the methods described in Examples 1-3, to provide pneumostasis at a lung injury site.

Tissue patches comprising fibrin-containing substrates (fabricated using the methods described in Examples 1 and 2) coated with adhesive (as described in Example 3) were evaluated via experiments on a pig following procedures outlined in Browdie, D. A., et al., “Tests of Experimental Tissue Adhesive Sealants,” Texas Heart Institute Journal, 2007, 34, pp. 313-317.

The surface of the lung was injured by picking up a small area of visceral pleura (the layer on the surface of the lung) and cutting away tissue with a Metzenbaum scissor such that the injury extended deep enough into the lung to create an obvious air leak and bleeding from the cut surface. The surface was sponged to remove blood, and an adhesive-coated tissue patch (allowing 2 cm of overlap onto adjacent intact visceral pleura) was applied. Pressure was held against the lung for 1 minute using a gauze sponge. The sponge was removed, leaving behind the adhered tissue patch. Bleeding was assessed by looking for escape of blood either from beneath the patch edges or through the body of the patch. Pneumostasis was assessed by submerging the lung in saline solution poured into the chest cavity, inflating the lung to pressures of at least 30 cm H₂O, and looking for air bubbles from the treated site.

This test described above was repeated at three distinct locations on the surface of the lung. Complete immediate and longer-term (2 hour) hemostasis and pneumostasis were observed at all sites.

### Example 6

Adhesion studies were performed using fibrin-containing substrates with substantially all water removed. The fibrin-containing substrates tested in this example were prepared by incubating fibrin-containing substrates fabricated according to the procedure outlined in Example 2 in air at 37° C. for 24 hours, producing a fibrin-containing substrate with substantially no moisture. Adhesion studies similar to those performed in Examples 4 and 5 have demonstrated that the patches with substantially all moisture removed performed as well as the patches with low levels (e.g., 6.3 wt % to 9.9 w %) of moisture.

### Example 7

This example describes the use of pericardial tissue as a substrate for a tissue adherent patch.

A porcine pericardium was harvested, and 2″×2″ squares were resected from the pericardial tissue. The pericardial tissue squares were dehydrated at room temperature for one minute in a beaker containing a mixture comprising 90 vol % of a pure ethanol dehydrating agent and 10 vol % of a glycerol humectant (99.5 vol % glycerol in 0.5 vol % water). The pericardium square was removed and blotted to remove excess glycerol/ethanol solution. Next, a 50/50 weight percentage blend of Carbopol® 974P NF and Noveon® AA-1 Polycarbophil acrylic acid polymers was mixed thoroughly. 625 mg of the mixed acrylic acid polymer powders was mixed with 1304 microliters of pure ethanol. The mixed powder/ethanol slurry was immediately spread onto the 2″×2″ pericardium tissue square. The pericardium tissue square was placed in a 37° C. incubator for 30 minutes. The resulting pericardium substrate with the polyacrylic acid solvent cast adhesive backing exhibited excellent adhesive properties.

### Example 8

This example describes the use of bowel tissue as a substrate for a tissue adherent patch.

A porcine small bowel was harvested, and 2″×2″ squares were resected from the tissue. The intestine tissue squares were dehydrated at room temperature for one minute in a beaker containing a mixture comprising 90 vol % of a pure ethanol dehydrating agent and 10 vol % of a glycerol humectant (99.5 vol % glycerol in 0.5 vol % water). The bowel tissue square was removed and blotted to remove excess glycerol/ethanol solution. Next, a 50/50 weight percentage blend of Carbopol® 974P NF and Noveon® AA-1 Polycarbophil acrylic acid polymers was mixed thoroughly. 625 mg of the mixed acrylic acid polymer powders was mixed with 1304 microliters of pure ethanol. The mixed powder/ethanol slurry was immediately spread onto the 2″×2″ small bowel tissue square. The intestine tissue square was placed in a 37° C. incubator for 30 minutes. The resulting intestine substrate with the polyacrylic acid solvent cast adhesive backing exhibited excellent adhesive properties.

### Example 9

This example describes the usage of starch-based material as a substrate for a tissue adherent patch.

15 grams of potato starch was added to 105 mL of water in a 250 mL beaker. 10 mL of glycerin was added to the water solution, and the contents were stirred to form an opaque solution. In a separate 50 mL beaker, 2.5 g of sodium bicarbonate were added to 30 mL of water. The starch solution in the 250 mL beaker was placed on a hot plate set at 130° C. and was stirred. The mixture was stirred until it because thick and almost transparent (about 5-7 min). Next, the solution of sodium bicarbonate and water was added to the starch solution, and the resulting mixture was stirred for an additional 1-2 min. The resulting mixture was then poured onto a baking pan in a thin sheet. The baking pan was placed in an oven to dry, after which, the starch sheet was removed. The corn starch sheets were cut into 2″×2″ squares.

Next, a 50/50 weight percentage blend of Carbopol® 974P NF and Noveon® AA-1 Polycarbophil acrylic acid polymers was mixed thoroughly. 625 mg of the mixed acrylic acid polymer powders was mixed with 1304 microliters of pure ethanol. The mixed powder/ethanol slurry was immediately spread onto the 2″×2″ starch square. The starch square was placed in a 37° C. incubator for 30 minutes. The resulting starch substrate with the polyacrylic acid solvent cast adhesive backing exhibited excellent adhesive properties.

### Example 10

This example illustrates the enhanced adhesive properties exhibited when an acrylic acid polymer is used in conjunction with a solvent.

A fibrin-containing substrate was fabricated according to the methods described in Example 1. The fibrin-containing substrate was then dehydrated in a liquid comprising a glycerol humectant and ethanol dehydrating agent, as described in Example 2.

625 mg of Sigma polyacrylic acid (average molecular weight of 3,000,000; Sigma product #306223) was mixed with 1304 μL of pure ethanol. The mixed slurry comprising ethanol and polyacrylic acid powder was immediately spread onto the dehydrated 2″×2″ fibrin-containing substrate. The fibrin-containing substrate was placed in an incubator and maintained at 37° C. for 30 minutes.

In a separate experiment, the Sigma polyacrylic acid powder was applied directly to a dehydrated fibrin-containing substrate.

The patch fabricated by applying the polyacrylic acid directly to the fibrin-containing substrate in powder form exhibited limited adhesion. The patch fabricated by applying the ethanol/polyacrylic acid slurry exhibited substantially better adhesion.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

What is claimed is:

1. A water-activated adhesive article, comprising:
a substrate; and
a water activated polymeric adhesive disposed within a solvent and in contact with the substrate.

2. The water-activated adhesive article of claim 1, wherein the substrate is biodegradable.

3. The water-activated adhesive article of any one of claims 1-2, wherein the substrate has a moisture content of less than about 20 wt %.

4. The water-activated adhesive article of any one of claims 1-3, wherein the substrate has a water content of less than about 20 wt %.

5. The water-activated adhesive article of any one of claims 1-4, wherein the substrate comprises a polymer.

6. The water-activated adhesive article of any one of claims 1-5, wherein the substrate comprises a biopolymer.

7. The water-activated adhesive article of any one of claims 1-6, wherein the substrate comprises a protein and/or a polysaccharide.

8. The water-activated adhesive article of any one of claims 1-7, wherein the substrate comprises fibrin, collagen, cellulose, starch, chitosan, hyaluronic acid, polylactic acid, polyglycolic acid and/or tissue-based materials.

9. The water-activated adhesive article of any one of claims 1-8, wherein the substrate comprises fibrin.

10. The water-activated adhesive article of any one of claims 1-9, wherein the substrate comprises a synthetic polymer.

11. The water-activated adhesive article of any one of claims 1-10, wherein the substrate is substantially free of thrombin.

12. The water-activated adhesive article of any one of claims 1-11, wherein the water-activated polymeric adhesive comprises the adhesive composition of any one of claims 1-18.

13. The water-activated adhesive article of any one of claims 1-12, wherein the solvent is a non-aqueous solvent.

14. The water-activated adhesive article of any one of claims 1-13, wherein the solvent is an organic solvent.

15. The water-activated adhesive article of any one of claims 1-14, wherein the solvent comprises an alcohol.

16. The water-activated adhesive article of any one of claims 1-15, wherein the solvent comprises at least one of methanol, ethanol, propanol, hexane, and ethyl acetate.

17. The water-activated adhesive article of any one of claims 1-16, wherein the solvent comprises ethanol.

18. The water-activated adhesive article of any one of claims 1-17, wherein the water-activated polymeric adhesive comprises at least one polymeric material comprising one or more monomers of formula (m-1); 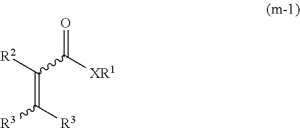
wherein:
X is O or NR¹;
each instance of R¹ is independently hydrogen or optionally substituted C₁₋₅₀alkyl; and
each instance of R² and R³ is independently hydrogen, optionally substituted C₁₋₄alkyl, or halogen.

19. The water-activated adhesive article of claim 18, wherein the at least one polymeric material comprising one or more monomers of formula (m-1) is cross-linked with one or more monomers of formula (m-2); 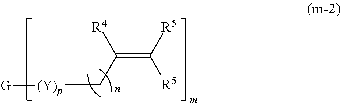
wherein:
each instance of R⁴ and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

20. The water-activated adhesive article of any one of claims 18-19, wherein the one or more monomers of formula (m-1) comprise a monomer selected from the group consisting of: 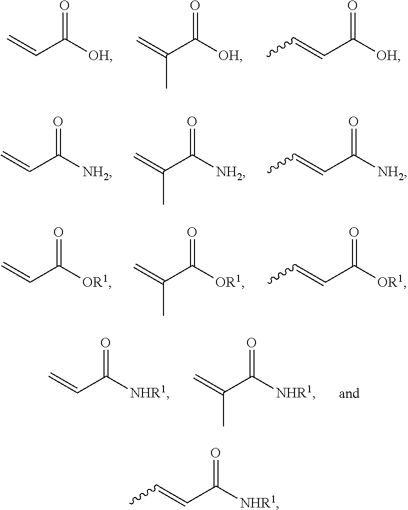
wherein R¹ is optionally substituted C₁₋₅₀alkyl.

21. The water-activated adhesive article of any one of claims 18-20, wherein the one or more monomers of formula (m-1) comprise:

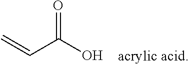

22. The water-activated adhesive article of any one of claims 18-21, wherein the one or more monomers of formula (m-1) comprise a monomer wherein X is O and R¹ is optionally substituted C₁₀₋₃₀alkyl.

23. The water-activated adhesive article of any one of claims 19-22, wherein m is an integer between 4 to 10, inclusive.

24. The water-activated adhesive article of any one of claims 19-22, wherein the one or more monomers of formula (m-2) is selected from the group consisting of:

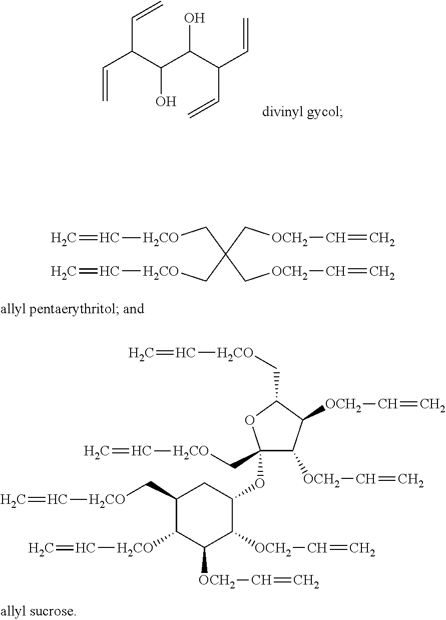

25. The water-activated adhesive article of any one of claims 18-24, wherein the polymeric material is a polymer of acrylic acid.

26. The water-activated adhesive article of claim 25, wherein the polymer of acrylic acid is cross-linked with allyl pentaerythritol or allyl sucrose.

27. The water-activated adhesive article of any one of claims 25-26, wherein the polymer of acrylic acid is cross-linked with divinyl glycol.

28. A method of applying an adhesive to a substrate, comprising:
applying an adhesive composition comprising a combination of a water-activated polymeric material and a solvent to a substrate.

29. The method of claim 28, wherein the water-activated polymeric material is at least partially dissolved in the solvent.

30. The method of any one of claims 28-29, wherein the water-activated polymeric material is at least partially dissolved in the solvent prior to applying the adhesive composition to the substrate.

31. The method of any one of claims 28-30, wherein the substrate is biodegradable.

32. The method of any one of claims 28-31, wherein the substrate has a moisture content of less than about 20 wt %.

33. The method of any one of claims 28-32, wherein the substrate has a water content of less than about 20 wt %.

34. The method of any one of claims 28-33, wherein the substrate comprises a polymer.

35. The method of any one of claims 28-34, wherein the substrate comprises a biopolymer.

36. The method of any one of claims 28-35, wherein the substrate comprises a protein and/or a polysaccharide.

37. The method of any one of claims 28-36, wherein the substrate comprises fibrin, collagen, cellulose, starch, chitosan, hyaluronic acid, polylactic acid, polyglycolic acid and/or tissue-based materials.

38. The method of any one of claims 28-37, wherein the substrate comprises fibrin.

39. The method of any one of claims 28-38, wherein the substrate comprises a synthetic polymer.

40. The method of any one of claims 28-39, wherein the substrate is substantially free of thrombin.

41. The method of any one of claims 28-40, wherein the solvent is a non-aqueous solvent.

42. The method of any one of claims 28-41, wherein the solvent is an organic solvent.

43. The method of any one of claims 28-42, wherein the solvent comprises an alcohol.

44. The method of any one of claims 28-43, wherein the solvent comprises at least one of methanol, ethanol, propanol, hexane, and ethyl acetate.

45. The method of any one of claims 28-44, wherein the solvent comprises ethanol.

46. The method of any one of claims 28-45, wherein the adhesive composition comprises the adhesive composition of any one of claims 1-18.

47. The method of claim 46, wherein the adhesive comprises at least one polymeric material comprising one or more monomers of formula (m-1); 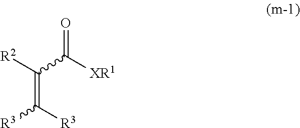
wherein:
X is O or NR¹;
each instance of R¹ is independently hydrogen or optionally substituted C₁₋₅₀alkyl; and
each instance of R² and R³ is independently hydrogen, optionally substituted C₁₋₄alkyl, or halogen.

48. The method of claim 47, wherein the at least one polymeric material comprising one or more monomers of formula (m-1) is cross-linked with one or more monomers of formula (m-2); 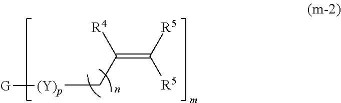
wherein:
each instance of R⁴ and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

49. The method of any one of claims 47-48, wherein the one or more monomers of formula (m-1) comprise a monomer selected from the group consisting of: 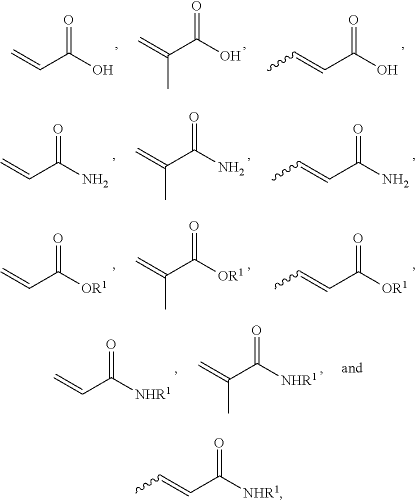
wherein R¹ is optionally substituted C₁₋₅₀alkyl.

50. The method of any one of claims 47-49, wherein the one or more monomers of formula (m-1) comprise:

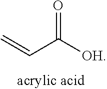

51. The method of any one of claims 47-50, wherein the one or more monomers of formula (m-1) comprise a monomer wherein X is O and R¹ is optionally substituted C₁₀₋₃₀alkyl.

52. The method of any one of claims 48-51, wherein m is an integer between 4 to 10, inclusive.

53. The method of any one of claims 48-52, wherein the one or more monomers of formula (m-2) is selected from the group consisting of:

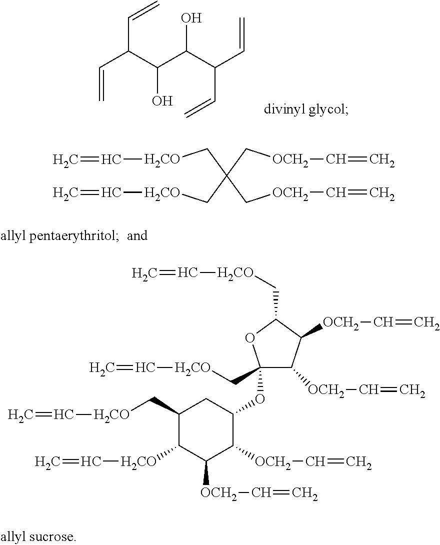

54. The method of any one of claims 47-53, wherein the polymeric material is a polymer of acrylic acid.

55. The method of claim 54, wherein the polymer of acrylic acid is cross-linked with allyl pentaerythritol or allyl sucrose.

56. The method of any one of claims 54-55, wherein the polymer of acrylic acid is cross-linked with divinyl glycol.

57. An adhesive composition, comprising:
(1) a first polymeric material comprising one or more monomers of formula (m-1) cross-linked with one or more monomers of formula (m-2); and
(2) a different, second polymeric material comprising one or more monomers of formula (m-1) cross-linked with one or more monomers of formula (m-2); 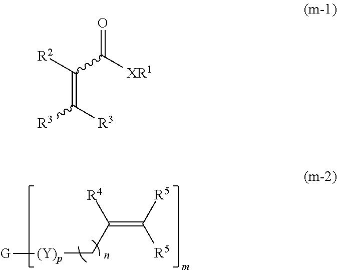
wherein:
X is O or NR¹;
each instance of R¹ is independently hydrogen or optionally substituted C₁₋₅₀alkyl;
each instance of R², R³, R⁴, and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

58. The composition of claim 57, wherein the one or more monomers of formula (m-1) comprise a monomer selected from the group consisting of: 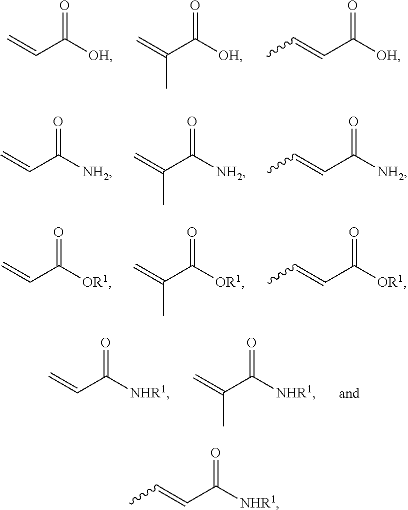
wherein R¹ is optionally substituted C₁₋₅₀alkyl.

59. The composition of claim 58, wherein the one or more monomers of formula (m-1) comprise:

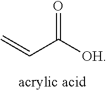

60. The composition of claim 57, wherein the one or more monomers of formula (m-1) comprise a monomer wherein X is O and R¹ is optionally substituted C₁₀₋₃₀alkyl.

61. The composition of claim 57, wherein m is an integer between 4 to 10, inclusive.

62. The composition of claim 57, wherein the one or more monomers of formula (m-2) is selected from the group consisting of:

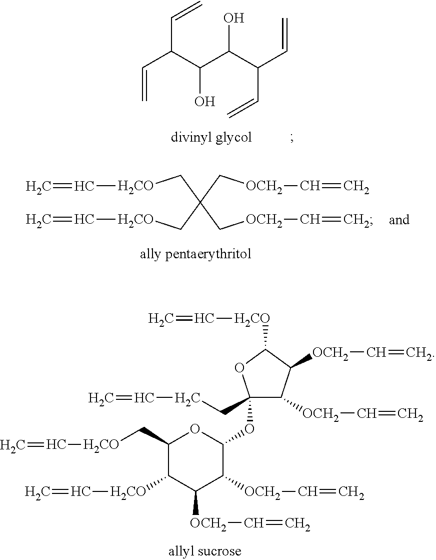

63. The composition of claim 57, wherein the first polymeric material is a polymer of acrylic acid cross-linked with allyl pentaerythritol or allyl sucrose.

64. The composition of claim 57, wherein the second polymeric material is a polymer of acrylic acid cross-linked with divinyl glycol.

65. The adhesive composition of any one of claims 57-64, wherein the first polymeric material is biodegradable.

66. The adhesive composition of any one of claims 57-65, wherein the second polymeric material is biodegradable.

67. The adhesive composition of any one of claims 57-66, wherein the first polymeric material and the second polymeric material are present within a liquid.

68. The adhesive composition of claim 67, wherein the liquid comprises a solvent in which at least one of the first and second polymeric materials are soluble.

69. The adhesive composition of claim 68, wherein the solvent is a non-aqueous solvent.

70. The adhesive composition of any one of claims 68-69, wherein the solvent is an organic solvent.

71. The adhesive composition of any one of claims 68-70, wherein the solvent comprises an alcohol.

72. The adhesive composition of any one of claims 68-71, wherein the solvent comprises at least one of methanol, ethanol, propanol, hexane, and ethyl acetate.

73. The adhesive composition of any one of claims 68-72, wherein the solvent comprises ethanol.

74. The adhesive composition of any one of claims 57-73, wherein the mass ratio of the first polymeric material to the second polymeric material is from about 1:10 to about 10:1.

75. A kit containing the adhesive composition of any one of claims 57-74.

76. A patch, comprising a solid matrix comprising fibrin and having a moisture content of less than 20 wt %.

77. The patch of claim 76, wherein the solid matrix is biodegradable.

78. The patch of any one of claims 76-77, wherein the solid matrix has a water content of less than about 20 wt %.

79. The patch of any one of claims 76-78, wherein the solid matrix has a fibrin content of at least about 50 wt %.

80. The patch of any one of claims 76-79, wherein the solid matrix is substantially free of thrombin.

81. The patch of any one of claims 76-80, comprising an adhesive material disposed over the patch.

82. The patch of any one of claims 76-81, wherein the adhesive material is substantially free of thrombin.

83. The patch of any one of claims 76-82, wherein the patch is in contact with an adhesive.

84. The patch of claim 83, wherein the adhesive comprises at least one polymeric material comprising one or more monomers of formula (m-1); 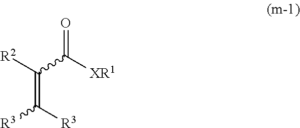
wherein:
X is O or NR¹;
each instance of R¹ is independently hydrogen or optionally substituted C₁₋₅₀alkyl; and
each instance of R² and R³ is independently hydrogen, optionally substituted C₁₋₄alkyl, or halogen.

85. The patch of claim 84, wherein the at least one polymeric material comprising one or more monomers of formula (m-1) is cross-linked with one or more monomers of formula (m-2); 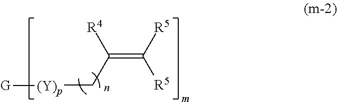
wherein:
each instance of R⁴ and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

86. The patch of any one of claims 84-85, wherein the one or more monomers of formula (m-1) comprise a monomer selected from the group consisting of: 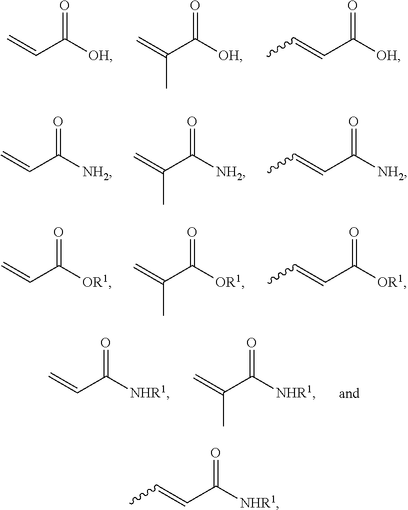
wherein R¹ is optionally substituted C₁₋₅₀alkyl.

87. The patch of any one of claims 84-86, wherein the one or more monomers of formula (m-1) comprise:

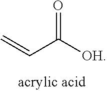

88. The patch of any one of claims 84-87, wherein the one or more monomers of formula (m-1) comprise a monomer wherein X is O and R¹ is optionally substituted C₁₀₋₃₀alkyl.

89. The patch of any one of claims 85-88, wherein m is an integer between 4 to 10, inclusive.

90. The patch of any one of claims 85-89, wherein the one or more monomers of formula (m-2) is selected from the group consisting of:

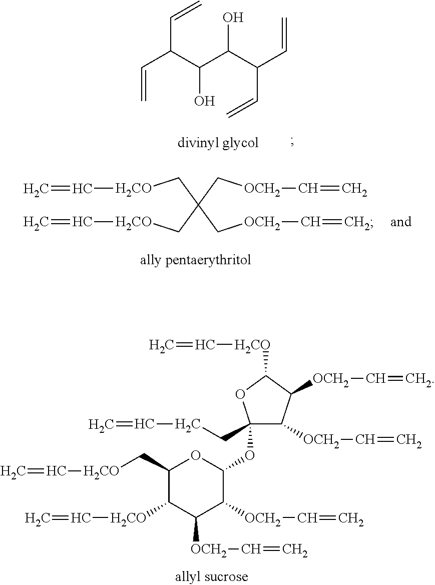

91. The patch of any one of claims 84-90, wherein the polymeric material is a polymer of acrylic acid.

92. The patch of claim 91, wherein the polymer of acrylic acid is cross-linked with allyl pentaerythritol or allyl sucrose.

93. The patch of any one of claims 91-92, wherein the polymer of acrylic acid is cross-linked with divinyl glycol.

94. The patch of any one of claims 83-93, wherein the adhesive comprises an adhesive composition of any one of claims 1-18.

95. A patch, comprising:
a solid matrix comprising fibrin,
wherein the solid matrix is substantially free of thrombin.

96. The patch of claim 95, wherein the solid matrix is biodegradable.

97. The patch of any one of claims 95-96, wherein the solid matrix has a moisture content of less than about 20 wt %.

98. The patch of any one of claims 95-97, wherein the solid matrix has a water content of less than about 20 wt %.

99. The patch of any one of claims 95-98, wherein the solid matrix has a fibrin content of at least about 50 wt %.

100. The patch of any one of claims 95-99, comprising an adhesive material disposed over the patch.

101. The patch of claim 100, wherein the adhesive material is substantially free of thrombin.

102. The patch of any one of claims 100-101, wherein the adhesive material comprises the adhesive composition of any one of claims 1-18.

103. The patch of any one of claims 100-102, wherein the adhesive comprises at least one polymeric material comprising one or more monomers of formula (m-1); 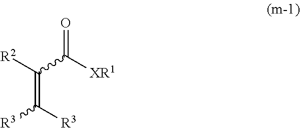
wherein:
X is O or NR¹;
each instance of R¹ is independently hydrogen or optionally substituted C₁₋₅₀alkyl; and each instance of R² and R³ is independently hydrogen, optionally substituted C₁₋₄alkyl, or halogen.

104. The patch of claim 103, wherein the at least one polymeric material comprising one or more monomers of formula (m-1) is cross-linked with one or more monomers of formula (m-2); 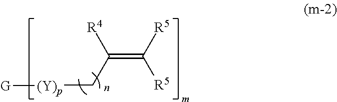
wherein:
each instance of R⁴ and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

105. The patch of any one of claims 103-104, wherein the one or more monomers of formula (m-1) comprise a monomer selected from the group consisting of: 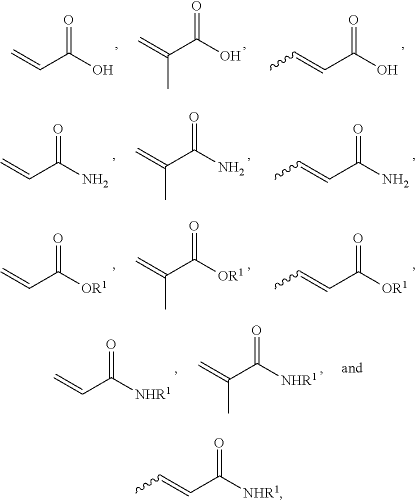
wherein R¹ is optionally substituted C₁₋₅₀alkyl.

106. The patch of any one of claims 103-105, wherein the one or more monomers of formula (m-1) comprise:

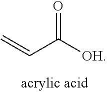

107. The patch of any one of claims 103-106, wherein the one or more monomers of formula (m-1) comprise a monomer wherein X is O and R¹ is optionally substituted C₁₀₋₃₀alkyl.

108. The patch of any one of claims 104-107, wherein m is an integer between 4 to 10, inclusive.

109. The patch of any one of claims 104-108, wherein the one or more monomers of formula (m-2) is selected from the group consisting of:

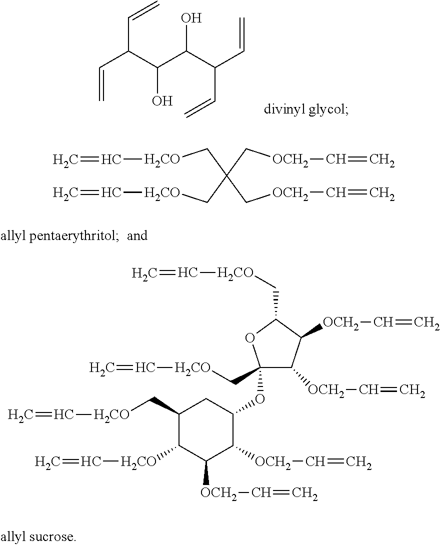

110. The patch of any one of claims 103-109, wherein the polymeric material is a polymer of acrylic acid.

111. The patch of claim 110, wherein the polymer of acrylic acid is cross-linked with allyl pentaerythritol or allyl sucrose.

112. The patch of any one of claims 110-111, wherein the polymer of acrylic acid is cross-linked with divinyl glycol.

113. A method of making a patch, comprising:
exposing a solid matrix comprising water and fibrin to a dehydrating agent such that water is removed from the solid matrix.

114. The method of claim 113, wherein the dehydrating agent comprises a dehydrating liquid.

115. The method of claim 114, wherein the dehydrating liquid comprises an alcohol.

116. The method of any one of claims 113-115, wherein the dehydrating agent comprises at least one of n-butanol, isopropanol, n-propanol, ethanol, methanol.

117. The method of any one of claims 113-116, wherein the dehydrating agent comprises ethanol.

118. The method of any one of claims 113-117, comprising exposing the solid matrix to a humectant.

119. The method of claim 118, wherein the humectant comprises at least one of propylene glycol, hexylene glycol, butylene glycol, glyceryl triacetate, neoagarobiose, a sugar alcohol, a polymeric polyol, quillaia, urea, aloe vera gel, MP diol, an alpha hydroxy acid, honey, and lithium.

120. The method of claim 119, wherein the sugar alcohol comprises at least one of glycerol, sorbitol, xylitol, and maltitol.

121. The method of claim 119, wherein the polymeric polyol comprises polydextrose.

122. The method of claim 119, wherein the alpha hydroxyl acid comprises lactic acid.

123. The method of any one of claims 118-122, wherein the humectant comprises an alcohol.

124. The method of any one of claims 118-123, wherein the humectant comprises glycerol.

125. The method of any one of claims 113-124, wherein, prior to exposing the solid matrix to the dehydrating agent, the solid matrix has a water content of at least about 50 wt %.

126. The method of any one of claims 113-125, wherein, after exposing the solid matrix to the dehydrating agent, the solid matrix has a water content of less than about 20 wt %.

127. The method of any one of claims 113-126, comprising, after exposing the solid matrix to the dehydrating agent, contacting the solid matrix with an adhesive composition.

128. The method of claim 127, wherein the adhesive composition comprises a water activated polymeric adhesive.

129. The method of any one of claims 127-128, wherein the adhesive composition comprises any of the adhesive compositions of claims 1-18.

130. The method of any one of claims 127-129, wherein the adhesive comprises at least one polymeric material comprising one or more monomers of formula (m-1); 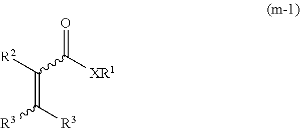
wherein:
X is O or NR¹;
each instance of R¹ is independently hydrogen or optionally substituted C₁₋₅₀alkyl; and
each instance of R² and R³ is independently hydrogen, optionally substituted C₁₋₄alkyl, or halogen.

131. The method of claim 130, wherein the at least one polymeric material comprising one or more monomers of formula (m-1) is cross-linked with one or more monomers of formula (m-2); 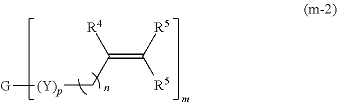
wherein:
each instance of R⁴ and R⁵ is independently hydrogen, optionally substituted C₁₋₄alkyl, or halogen;
G is optionally substituted aliphatic, optionally substituted heteroaliphatic, optionally substituted aryl, optionally substituted heteroaryl, a polymer, or a carbohydrate;
Y is O or NH;
p is 0 or 1;
n is 0 or 1, provided when p is 1, then n is 1; and
m is an integer of 2 or greater.

132. The method of any one of claims 130-131, wherein the one or more monomers of formula (m-1) comprise a monomer selected from the group consisting of: 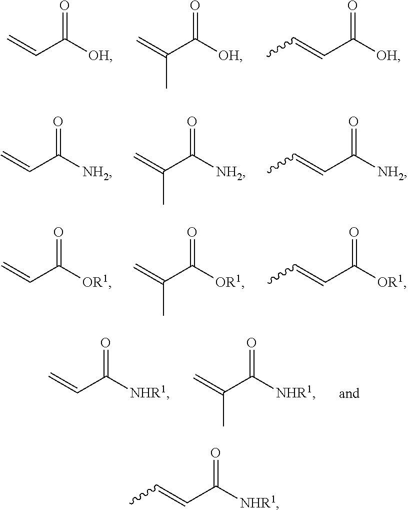
wherein R¹ is optionally substituted C₁₋₅₀alkyl.

133. The method of any one of claims 130-132, wherein the one or more monomers of formula (m-1) comprise:

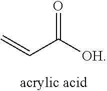

134. The method of any one of claims 130-133, wherein the one or more monomers of formula (m-1) comprise a monomer wherein X is O and R¹ is optionally substituted C₁₀₋₃₀alkyl.

135. The method of any one of claims 131-134, wherein m is an integer between 4 to 10, inclusive.

136. The method of any one of claims 131-135, wherein the one or more monomers of formula (m-2) is selected from the group consisting of:

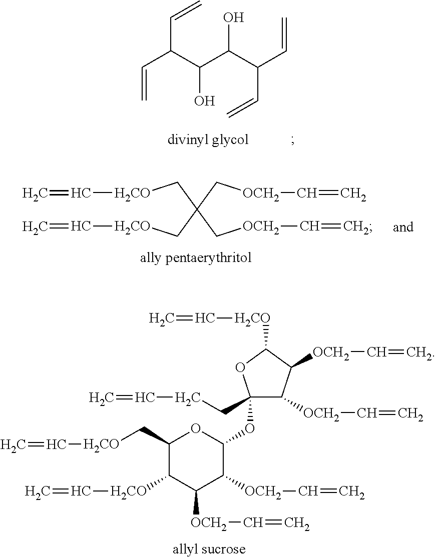

137. The method of any one of claims 130-136, wherein the polymeric material is a polymer of acrylic acid.

138. The method of claim 137, wherein the polymer of acrylic acid is cross-linked with allyl pentaerythritol or allyl sucrose.

139. The method of any one of claims 137-138, wherein the polymer of acrylic acid is cross-linked with divinyl glycol.

140. A method of preparing a solid matrix comprising cross-linked fibrin, comprising:
applying a compressive force to a liquid containing composition comprising fibrin and/or fibrinogen;
passing at least a portion of a liquid component of the composition through a filter so that at least a portion of the fibrin and/or fibrinogen is separated from the at least a portion of the liquid component; and
polymerizing the fibrinogen to form fibrin and/or cross-linking the fibrin to form the solid matrix comprising cross-linked fibrin,
wherein the fibrin and fibrinogen are not exposed to substantial amounts of thrombin during the preparation of the patch.

141. A method of preparing a solid matrix comprising cross-linked fibrin, comprising:
applying a compressive force to a liquid containing composition comprising fibrin and/or fibrinogen within a chamber; and
polymerizing the fibrinogen to form fibrin and/or cross-linking the fibrin to form the solid matrix comprising cross-linked fibrin,
wherein the fibrin and fibrinogen are not exposed to substantial amounts of thrombin during the preparation of the patch.

142. The method of any one of claims 140-141, comprising exposing the solid matrix to a dehydrating agent such that water is removed from the solid matrix

143. The method of claim 142, wherein the dehydrating agent comprises a dehydrating liquid.

144. The method of claim 143, wherein the dehydrating liquid comprises an alcohol.

145. The method of any one of claims 142-144, wherein the dehydrating agent comprises at least one of n-butanol, isopropanol, n-propanol, ethanol, methanol.

146. The method of any one of claims 142-145, wherein the dehydrating agent comprises ethanol.

147. The method of any one of claims 140-146, comprising exposing the solid matrix to a humectant.

148. The method of claim 147, wherein the humectant comprises at least one of propylene glycol, hexylene glycol, butylene glycol, glyceryl triacetate, neoagarobiose, a sugar alcohol, a polymeric polyol, quillaia, urea, aloe vera gel, MP diol, an alpha hydroxy acid, honey, and lithium.

149. The method of claim 148, wherein the sugar alcohol comprises at least one of glycerol, sorbitol, xylitol, and maltitol.

150. The method of claim 148, wherein the polymeric polyol comprises polydextrose.

151. The method of claim 148, wherein the alpha hydroxyl acid comprises lactic acid.

152. The method of any one of claims 147-151, wherein the humectant comprises an alcohol.

153. The method of any one of claims 147-152, wherein the humectant comprises a sugar alcohol.

154. The method of any one of claims 147-153, wherein the humectant comprises glycerol.

155. The method of any one of claims 142-154, wherein, prior to exposing the solid matrix to the dehydrating agent, the solid matrix has a water content of at least about 50 wt %.

156. The method of any one of claims 142-155, wherein, after exposing the solid matrix to thedehydrating agent, the solid matrix has a water content of less than about 20 wt %.

157. The method of any one of claims 142-156, comprising, after exposing the solid matrix to the dehydrating agent, contacting the solid matrix with an adhesive composition.

158. The method of claim 157, wherein the adhesive composition comprises a water activated polymeric adhesive.

159. The method of any one of claims 157-158, wherein the adhesive composition comprises any of the adhesive compositions of claims 1-18.

160. The method of any one of claims 140-159, wherein the liquid containing composition comprising fibrin and/or fibrinogen comprises a plasma component of whole blood.

161. The method of any one of claims 140-160, wherein the liquid containing composition comprising fibrin and/or fibrinogen comprises a plasma component of human blood, a plasma component of equine blood, a plasma component of bovine blood, and/or a plasma component of porcine blood.
